(19) United States
(12) Patent Application Publication
(10) Pub. No.: US 2004/0019192 A1
(43) Pub. Date: Jan. 29, 2004

(54) Process for modifying plants

(76) Inventors: Mark Harker, Shambrook (GB); Susan Amanda Hellyer, Shambrook (GB); Niklas Holmberg, Shambrook (GB); Dick Safford, Shambrook (GB)

Correspondence Address: UNILEVER, PATENT DEPARTMENT, 45 RIVER ROAD, EDGEWATER, NJ, 07020, US

(21) Appl. No.: 10/432,267

(22) PCT Filed: Nov. 7, 2001
(86) PCT No.: PCT/EP01/13037

(30) Foreign Application Priority Data
Nov. 24, 2000 (EP) 003104031

(51) Int. Cl. C12Q 1/68; C07H 21/02; C07H 21/04

(52) U.S. Cl. 536/23.1; 435/6

(57) ABSTRACT

The use of a gene expressing a non-feed back inhibited HMG-reductase in combination with a gene expressing sterol methyltransferasel to increase the level of sterols in plants.

### FIELD OF INVENTION

The invention relates to a process for the modification of plants, more specifically a process for increasing the isoprenoid levels in plants.

### BACKGROUND OF THE INVENTION

Many approaches have been suggested for modifying the isoprenoid production in plants.

Whereas only a few sterols exist in animals, with cholesterol being by far the major one, in plants a wide range of sterols are found. Structural variations between these arise from different substitutions in the side chain and the number and position of double bonds in the tetracyclic skeleton.

Plant sterols can be grouped by the presence or absence of one or more functionalities. For example they can be divided into three groups based on methylation levels at C4 as follows: 4-desmethylsterols or end product sterols, 4α-monomethylsterols and 4,4-di-methylsterols. Naturally occurring 4-desmethylsterols include sitosterol, stigmasterol, brassicasterol, Δ7-avenosterol and campesterol.

In most higher plants, sterols with a free 3β-hydroxyl group (free sterols) are the major end products. However sterols also occur as conjugates, for example, where the 3-hydroxy group is esterified by a fatty acid chain, phenolic acids or sugar moieties to give sterol esters. For the purpose of this description the term sterol refers both to free sterols and conjugated sterols. However in this specification references to levels, amounts or percentages of sterol refer to the total weight sterol groups whereby the weight of the conjugating groups such as fatty acid, phenolic acid or sugar groups is excluded.

To date most studies aimed at manipulating sterols in plants have involved other than 4-desmethylsterols with the purpose of increasing resistance to pests or to fungicides.

WO 98/45457 describes the modulation of phytosterol compositions to confer resistance to insects, nematodes, fungi and/or environmental stresses, and/or to improve the nutritional value of plants by using a double stranded DNA molecule comprising a promoter, a DNA sequence encoding a first enzyme which binds a first sterol and produces a second sterol and a 3′ non-translated region which causes polyadenylation at the 3′ end of the RNA. Preferably the enzyme is selected from the group consisting of S-adenosyl-L-methionine-Δ²⁴⁽²⁵⁾-sterol methyl transferase, a C-4 demethylase, a cycloeucalenol to obtusifoliol-isomerase, a 14-α-demethylase, a Δ⁸ to Δ7- isomerase, a Δ7-C-5-desaturase and a 24,25-reductase.

U.S. Pat. No. 5,306,862 describes a method of increasing sterol accumulation in a plant by increasing the copy number of a gene encoding a polypeptide having HMG-COA reductase activity to increase the resistance of plants to pests.

Similarly U.S. Pat. No. 5,349,126 discloses a process to increase the squalene and sterol accumulation in transgenic plants by increasing the amount of a gene encoding a polypeptide having HMG-CoA reductase activity to increase the pest resistance of transgenic plants.

WO 97/48793 discloses a C-14 sterol reductase polypeptide for the genetic manipulation of a plant sterol biosynthetic pathway.

WO 96/09393 discloses a DNA sequence encoding squalene synthetase.

WO 97/34003 discloses a process of raising squalene levels in plants by introduction into a genome of a plant a DNA to suppress expression of squalene epoxidase.

WO 93/16187 discloses new plants containing in its genome one or more genes involved in the early stages of phytosterol biosynthesis, preferably the genes encode mevalonate kinase.

U.S. Pat. No. 5,589,619 discloses accumulation of squalene in plants by introducing a HMG-COA reductase gene to increase production of sterol and resistance to pests. Example 10 discloses increased squalene levels in the seeds of these plants.

WO 00/08190 discloses a DNA sequence encoding a sterol methyltransferase isolated from Zea mays.

In plants, mevalonate synthesis via 3-hydroxy-3-methylglutaryl Coenzyme A reductase (HMGR) is one of the steps in isoprenoid biosynthesis.

Gondet et al in Plant Physiology (1994) 105:509-518 has isolated a tobacco mutant showing dramatically altered sterol compositions in leaf tissue with significant increases in the proportion of cyclopropylsterols and HMGR activities increased by approximately 3-fold.

Re et al in The Plant Journal (1995) 7(5), 771-784 have shown that the over-expression of Arabidopsis thaliana HMG CoA reductase (HMG 1) is not sufficient to alter the bulk synthesis and accumulation of end products of the plant isoprenoid pathway.

Applicants believe that the reason for this is that the activity of HMGR in plants is subject to feedback inhibition by sterols. Some HMGR genes, however, are non-feed back inhibited. Examples of such genes are non-plant HMGR genes lacking the membrane-binding domain, such as the truncated hamster HMGR genes or the truncated Saccharomyces cerevisiae genes, and HMGR genes (or truncated versions thereof) from high isoprenoid producing plants such as Hevea brasiliensis.

A truncated hamster HMGR gene, lacking the membrane-binding domain, was expressed in tobacco plants under the control of the CaMV 35S promoter (Chappell et al., Plant Physiology (1995) 109: 1337-1343). This resulted in a 3- to 6-fold increase in total HMGR activity in leaf tissue.

Schaller et al in Plant Physiology (1995) 109:761-770 discloses the introduction of the hmg1 gene from Hevea brasiliensis into tobacco leading to an enhanced sterol production, especially of cycloartenol, in leaf tissue.

Polakowski et al in Applied Microbial Biotechnology (1998) 59:66-71 describes the use of a truncated Saccharomyces cerevisiae hmg 1 gene in yeast, leading to the accumulation of squalene.

In plants, 24-methylene cycloartanol production from cycloartenol via sterol methyltransferasel (SMT1) is one of the steps in isoprenoid biosynthesis.

Bouvier-Nav et al in Bur. J. Biochem. 256, 88-96 (1988) describes two families of sterol methyl transferases (SMTs), The first (SMT1) applying to cycloartenol and the second (SMT2) to 24-methylene lophenol.

Schaller et al in Plant Physiology (1998) 118: 461-169 describes the over-expression of SMT2 from Arabidopsis in tobacco resulting in a change in the ratio of 24-methyl cholesterol to sitosterol in the tobacco leaf.

Diener et al in The Plant Cell (2000) 12: 853-870 describes the functional characterisation of an Arabidopsis SMT1 gene and show that mutants lacking the gene display poor growth and fertility.

Schaeffer et al in Lipids (2000) 35: 263-269 describe the effects of expressing Nicotiana tabacum SMT1 and SMT2 genes in transgenic tobacco. Overexpression of SMT1 results in variations in the level of cycloartenol and concomitant changes in the proportion of 24-ethyl sterols. Over expression of SMT 2 alters the ratio of 24-methyl cholesterol to sitosterol resulting in reduced growth.

Surprisingly it has now been found that expressing genes encoding specific HMG-reductase enzymes in combination with those encoding sterol methyltransferasel can advantageously be used to further increase the nutritional value of plants especially in the seeds thereof.

Surprisingly it has been found that the use of non-feedback regulated HMGR in combination with overexpression of sterol methyltransferasel leads to the further enhancement of nutritionally beneficial sterol for example in the seeds of said plants compared to plants where only one of the above genes has been expressed.

The present invention aims to modify sterol levels in plants, especially the seeds of plants whereby this modification can either involve an increase of the level of (beneficial) sterols or a decrease of the level of (less-desired) cholesterol.

The present invention aims to increase sterol levels in plants, whereby the sterols are preferably nutritionally attractive 4-desmethylsterols such as sitosterols, stigmasterols, brassicasterol, Δ7-avenosterol or campesterols and whereby the sterols are expressed in the seeds.

### STATEMENT OF THE INVENTION

Accordingly the invention relates to the use of a gene expressing a SMT1 in combination with a non feedback inhibited HMGR gene to increase the level of sterols in plant tissue and/or decrease the level of cholesterol in plant tissue.

In another aspect, the invention relates to a modified plant having incorporated into its genome one or more genes for increasing the expression of SMT1 and increasing the expression of non-feedback inhibited HMGR.

### DETAILED DESCRIPTION OF THE INVENTION

In higher plants, isoprenoids are a large family of compounds with diverse roles. They include sterols, the plant hormones gibberellins and abscisic acid, components of photosynthetic pigments, phytoalexins and a variety of other specialised terpenoids.

Sterols, especially 4-desmethylsterols are of interest because they contribute to the nutritional quality, flavour and colour of fruits and vegetable oils. Of particular interest are isoprenoid compounds of nutritional benefit such as fat-soluble sterols. These may be efficacious in reducing coronary heart disease, for example, some phytosterols have been shown to lower serum cholesterol levels when increased in the diet and vitamin E reduces atherosclerotic plaques via decreased oxidation of LDL.

Expression of such compounds in plant seeds in particular in oilseeds is commercially advantageous as generally the harvesting of such ingredients from seeds is very convenient and, in some instances, it may be possible to extract the oil in combination with the sterols from the seed, leading to an oil containing elevated levels of sterol without or with the reduced need for separate addition of sterols.

Preferred sterols are 4-desmethylsterols, most preferred sitosterol, stigmasterol, brassicasterol, avenosterol and campesterol. Also preferably, at least part of the sterols, for example at least 70 wt % based on the total of the sterols in the seed are esters of sterols with C10-24 fatty acids. In a very preferred embodiment the sterols comprise C10-24 esters of 4-desmethylsterols.

As discussed above, several approaches have been suggested to alter levels of isoprenoids in plants.

It has now been found that for the enhancement of isoprenoid levels in plants particularly in the seeds thereof an even more preferred route is to use a non-feedback inhibited HMGR gene in combination with sterol methyltransferasel. The use of such a combination of genes is especially advantageous to enhance the levels of 4-desmethylsterols, more so than expression of either gene singularly. Even more preferred, the use of such genes enhances the level of stigmasterol, sitosterol and campesterol in seeds. Also the use of such genes is especially advantageous to enhance the levels of isoprenoids in oilseeds containing more than 10 wt % based on dry weight of triglycerides.

In a first embodiment of the invention the non-feed back inhibited HMG reductase is an enzyme which is expressed by a truncated non-plant HMGR gene, said truncation preferably leading to an enzyme lacking the membrane binding domain, but whereby the HMGR functionality of the gene is preferably maintained. Examples of such genes are the truncated hamster or yeast HMGR genes.

A second-preferred-embodiment of a non-feedback inhibited HMG reductase is an enzyme expressed by HMGR genes from high isoprenoid producing plants such as Hevea brasiliensis. Especially preferred are truncated versions of HMGR produced by genes from high isoprenoid producing plants such as Hevea brasiliensis, most preferred truncated versions are used whereby said HMGR lacks the membrane binding domain.

The intact HMGR enzyme comprises three regions: a catalytic region, containing the active site of the enzyme, a membrane binding region, anchoring the enzyme to the endoplasmic reticulum and a linker region joining the catalytic and membrane binding regions of the enzyme. The membrane-binding domain occupies the N-terminal region of the enzyme, whereas the catalytic region occupies the C-terminal region. It is believed that feedback inhibition in most plants generally requires the presence of the membrane-binding region of the enzyme. Therefore a preferred embodiment of the invention relates to the use of an HMGR gene expressing an enzyme with an inactivated or without a membrane binding domain, whereby said gene is preferably used to increase the level of 4-desmethylsterols in plant tissue such as the seeds of plants.

An example of HMG reductase with an inactivated or without a membrane binding domain is the HMG reductase expressed by the truncated hamster HMGR gene as described by Chappell (see above). The truncation is believed to remove the membrane binding domain from the HMG reductase whereafter a significant reduction of feedback inhibition occurs. Other truncated or mutated genes whereby the membrane binding domain is removed or inactivated can equally be used. An example of this is the truncated HMGR gene as used by Polakowski (see above).

Preferred examples of HMG reductases are those expressed by HMGR genes obtained from plants which naturally have the tendency to develop high levels of isoprenoids such as for example triterpenes and rubber. Examples of such plants are Asteraceae, especially Euphorbiaceae. Therefore another preferred embodiment of the invention relates to the use of an HMGR gene isolated from Asteraceae to increase the level of sterols, particularly 4-desmethylsterols in plant tissue, particularly the seeds of plants. Preferably the HMGR gene is isolated from Hevea brasiliensis. Especially preferably truncated versions of such plant genes may be used. A specific promoter can be inserted into the plant genome to ensure that the HMGR gene is upregulated, preferably within the seed tissue of the plant.

Suitably the SMT1 gene can be naturally present in the plant. In accordance to the invention the circumstances are then altered such that increased expression of SMT1 preferably in the seed region of the plant will take place. Possible ways to do this may be to upregulate facilitating molecules e.g. such as transcription factors. Alternatively, a specific promoter can be inserted into the plant genome to ensure that the SMT1 gene is upregulated. Alternatively, the copy number of the “homologous” SMT1 gene may be increased to increase the expression thereof.

Alternatively, the SMT1 gene can be a heterologous gene, for example derived from other plant or microbial sources. For example, the SMT1 gene may be derived from Arabidopsis, tobacco or yeast.

Cholesterol is a less desired component of food products because consumers have a desire to reduce their cholesterol consumption. It is believed that reduced serum cholesterol levels lead to a reduced risk of cardiovascular disease. Therefore, in one embodiment the invention relates to the reduction of the cholesterol level in plant tissue, especially the seeds of plants.

As discussed above, several approaches have been suggested to alter the levels of isoprenoids and/or cholesterol in plants. It has now been found that for the enhancement of isoprenoid levels in seeds a preferred route is to use a SMT1 gene. The use of such genes is especially advantageous to enhance the levels of 4-desmethylsterols, even more preferred the level of stigmasterol, sitosterol, brassicasterol, isofucosterol and campesterol in seeds. Also, the use of such genes is especially advantageous to enhance the levels of isoprenoids in oilseeds containing more than 10 wt % based on dry weight of triacylglycerols.

The invention also provides a method of transforming a plant by

A1s) transforming a plant cell with a recombinant DNA construct comprising a DNA segment encoding a polypeptide with non feedback inhibited HMGR activity and a polypeptide encoding a sterol methyltransferasel activity and promoters for driving the expression of said polypeptides in said plant cell to form a transformed plant cell; or

A2) re-transforming a plant cell expressing a non-feedback inhibited HMGR activity with a gene encoding a sterol methyltransferasel activity; or

A3) re-transforming a plant cell expressing a sterol methyltransferasel activity with a gene encoding a non-feedback inhibited HMGR activity; and

B) regenerating the above transformed plant cells into transgenic plants; and

C) selecting transgenic plants that have enhanced levels of 4-desmethylsterols compared to wild type strains of the same plant.

DNA segments encoding non-feedback inhibited HMGR or sterol methyltransferasel, for use according to the present invention, may suitably be obtained from animals, microbial sources or plants. Alternatively, equivalent genes could be isolated from gene libraries, for example by hybridisation techniques with DNA probes.

The invention will now further be illustrated in the following examples:

### EXAMPLE 1

### Co-Expression of Hevea brasiliensis hmg1 and Nicotiana tabacum SMT1 in Plants

E. coli strain DH5 (Gibco BRL) was used as the host strain in all cloning and sub-cloning procedures. Binary vector pSJ34 (PCT/EP/00/09374) was created by filling in the BamHI site of pGPTV-Kan[Becker et al Plant Mol Biol (1992) 20:1195-97], between the selectable marker and the p(A)g7 3′-end, with Klenow enzyme. The construction of plasmids pNH6 and pNH8 have been described in our non-pre-published patent applications PCT/EP00/09374 and EP 00303193.7 respectively. Bacteria were cultivated in LB medium (10 g/l tryptone, 5 g/l yeast extract, 5 g/l NaCl) supplemented with the appropriate selection pressure (ampicillin 100 μg/ml or kanamycin 50 μg/ml) on a rotary shaker (210 rpm) at 37° C.

Restriction endonucleases, T4 DNA ligase, shrimp alkaline phosphatase and molecular markers (X, XIV and XVII) were purchased from Roche. The enzymes were used according to the suppliers' recommendations. All chemicals and reagents used were of analytical grade and available from Fisher Scientific UK, Sigma or BDH. The following oligonucleotide primers were used: F72, 5′-GCC ATA ATA CTC GAA CTC AG-3′; 35S, 5′-TCC ACT GAC GTA AGG GAT GAC-3′; CERV1S, 5′-GTC TGT CTA AAG TAA AGT AGA TGC G-3′; NOSAS, 5′-CCG GCA ACA GGA TTC AAT CTT-3′.

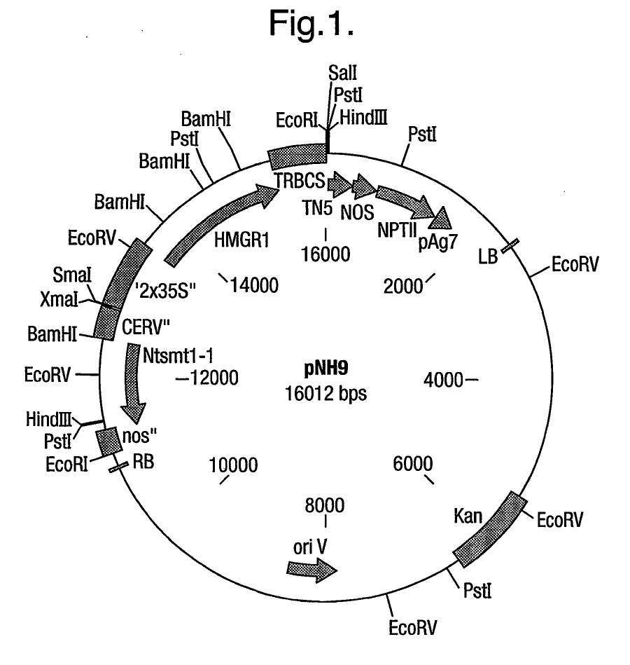

The Qiagen mini prep kit was used to obtain plasmid DNA for sequencing and sub-cloning procedures. The Qiagen gel extraction kit was used to purify DNA from agarose gels. Plasmid pNH6 was digested with XmaI and EcoRI and plasmid pNH8 with XmaI and SalI releasing the CERV-Ntsmt1-NOS and double CaMV35S-Hevea hmg1-TRBCS cassettes, respectively. The digestion reactions were separated in an agarose gel and the expression cassettes were excised and purified. Binary vector pSJ34 was digested with EcoRI and SalI, purified and subsequently treated with shrimp alkaline phosphate to remove the terminal phosphate groups. Using a three-way ligation, both expression cassettes were inserted into pSJ34 resulting in pNH9 (FIG. 1). First PCR, using gene specific primers, and second restriction enzyme digestion was used to select positive clones. Positive clones were sequenced confirming the integrity of the junctions between transgene and terminator.

Transformation of Tobacco with Binary Vectors

Electrocompetent Agrobacterium tumefaciens cells (strain LBA4404) were defrosted on ice and 5 ng of vector plasmid added. Cells plus plasmid were then placed into a pre-chilled electroporation cuvette and electroporated in a Bio Rad Gene Pulser at a capacitance of 25 μF and at 600 ohms. Immediately after electroporation 950 μl of 2×TY broth was added, the cells mixed gently and placed in a sterile vial. The cells were shaken at 28° C. for 2 hours and 25 μl aliquots plated on solid Lennox media containing rifampicin 50 μg/ml and kanamycin 50 μg/ml and incubated at 28° C. for 3 days. Single colonies were used to inoculate 10 μl of water (for PCR confirmation) and 500 μl of Lennox media containing rifampicin 50 μg/ml and kanamycin 50 μg/ml.

PCR positive cultures were used to inoculate a 10 ml of Lennox media broth containing rifampicin 50 μg/ml and kanamycin 50 μg/ml. The overnight culture was spun down at 3000 g and resuspended in an equal volume of MS media (3% sucrose). Leaf segments were cut from young tobacco leaves from plants grown in tissue culture. Segments were placed directly into the agrobacterium solution and left for 10 minutes. The segments were then removed and placed upper surface down on feeder plates (10 per plate) and left for 2 days in low light at 22° C. The leaf segments were placed, upper surface up, on tobacco shooting media with hormones containing cefotaxime 500 μg/ml and kanamycin 50 μg/ml and placed in a growth room at 24° C. with a 16 hrs light/8 hrs dark regime. Three weeks later, the callusing segments were transferred to Magenta tubs containing tobacco shooting media. Once formed, shoots were excised and placed on tobacco shooting media containing cefotaxime 500 μg/ml and kanamycin 50 μg/ml without hormones, to root. Rooted plants were then potted up into a 50% perlite/50% compost mixture and placed in a propagator. After 1 week the plants were removed from the propagator and subsequently potted up into 5 inch pots. Once flowering had began paper bags were placed over the flowers to prevent cross pollination. When flowering had finished and pods formed the bags were removed and mature pods harvested. Mature leaves and seed from dry pods were harvested and stored for subsequent analysis.

Sterol Analysis

For sterol analysis, the plant tissue obtained as above is freeze-dried, then ground to a fine powder. 250 μl of 0.2% w/v dihydrocholesterol dissolved in chloroform is pipetted into a screw-top septum vial. After removal of solvent, an amount of the plant tissue (50 mg) is added to the vial, and total lipid extracted with 5 ml of a 2:1 v/v mixture of chloroform: methanol. The vial is capped and placed in a hot block maintained at 80-85° C. After 30 minutes the contents are filtered and the vial is washed out with a second 5 ml aliquot of the chloroform: methanol mixture. The contents of the vial are filtered once more and the filtrates combined. The solvent portion of the filtrate is blown off using a stream of nitrogen gaq to isolate the lipid residue.

The lipid fraction is then subjected to transmethylation by heating at 80-85° C. in 1 ml of toluene and 2 ml of 0.5N sodium methoxide in methanol. After 30 minutes, 2 ml of a 14% boron trifluoride solution in methanol is added and heated for a further 10 minutes at 80-85° C. After cooling, 2-3 ml of diethyl ether followed by 5 ml of deionised water are added. The ether fraction is removed and a further ether extraction carried out. The ether fractions are combined, backwashed with approx. 5 ml of water and dried overnight over anhydrous sodium sulphate. The ether phase is filtered and the solvent removed using a stream of nitrogen gas.

Sterols are dissolved in 300-400 μL of toluene and silylated by the addition of 200 μl of 95:5 N,O-bis(trimethylsilyl)acetamide:trimethylchlorosilane followed by incubation at 50° C. for 10 minutes. GC analysis is carried out using a 25 m×0.32 mm i.d. (0.25 um film thickness) 5% BPX5 column (ex SGE) in a Perkin-Elmer 8420 GC. The temperature program is 180-240° C. at 10° C./min, followed by 240-355° C. at 15° C./min. and, finally, 5 min. at 355° C. The FID temperature is 380° C. and the helium pressure 10 psi. A volume of 1.0 μl is injected onto the column. A GC response factor of 1.0 for each of the sterols with respect to the dihydrocholesterol internal calibrant is assumed.

Table 1 shows the sterol analysis of leaf samples obtained from tobacco transformed with the NH9 vector co-expressing a full length Hevea HMGR and tobacco SMT1. Leaves from 12 independent transgenic plants (NH9) were analysed along with leaves from 6 independent untransformed plants (SR1) which had been generated via tissue culture, and leaves from 5 independent plants transformed with control vector lacking the gene of interest (pVEC). The total sterol content of the SR1 control leaves ranged from 0.165-0.268% dry weight and those of the pVEC controls from 0.175%-0.269% dry weight. The NH9 transgenic leaves contained total sterol contents ranging from 0.176-0.318% dry weight, representing increases of up to 36.4% over the mean SR1 sterol content and 45.6% over the mean of ‘beneficial’ 4-desmethylsterols (4-desmethylsterols minus cholesterol). Also of note are the dramatically reduced levels of cholesterol in the NH9 samples, with 6 of the 12 samples having zero (or below detection) levels of cholesterol.

Table 2 shows the sterol analysis of mature seed samples from tobacco transformed with the NH9 vector co-expressing full length Hevea HMGR and tobacco SMT1. Seeds from 27 independent transgenic plants (NH9) were analysed along with seeds from 12 SR1 and 6 pVEC control plants. Seeds from the control SR1 plants contained total sterol contents ranging from 0.339-0.425% dry weight and those of the pVEC control plants from 0.301-0.413% dry weight. Seeds from the NH9 transgenic plants contained total sterol contents ranging from 0.307-0.545% representing increases of up to 43.0% over the mean SR1 control total sterol value and up to 51.6% over the mean SR1 beneficial 4-desmethylsterol value. Significant decreases in the level of cycloartenol, the substrate for the sterol methyl transferasel, were found in the high sterol NH9 samples. Cholesterol levels in the high sterol NH9 samples were also significantly reduced. Of particular note are the higher levels of sitosterol in the high sterol NH9 lines compared to control levels.

### EXAMPLE 2

### Co-Expression of a Truncated Form of Hevea brasiliensis hmg1 and Nicotiana tabacum SMT1 in Plants

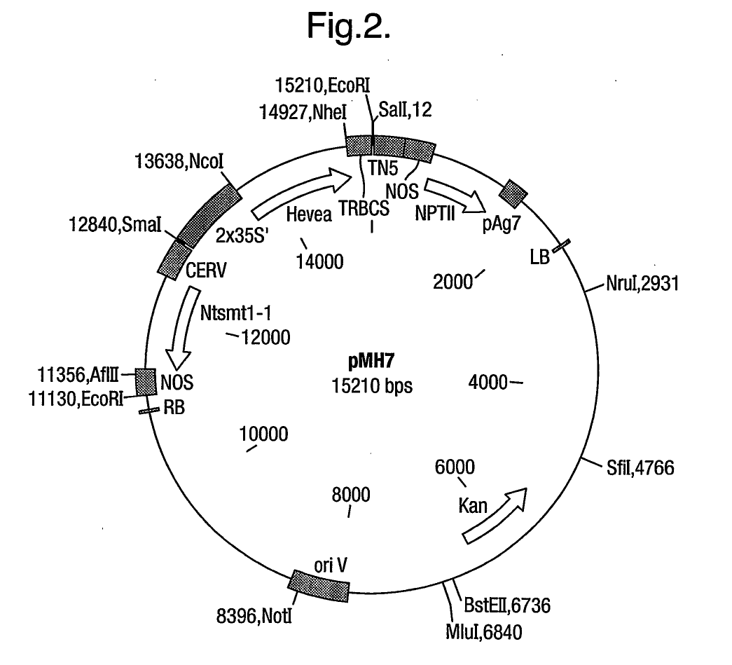

A truncated form of Hevea HMGR, lacking the N-terminal membrane-binding domain, was cloned using the Hevea brasiliensis hmg1 as template. The Hevea brasiliensis (H.B.K.) Müll. Arg. thmg1 was cloned using the primers based on the published sequence [Chye et al (1991) Plant Mol Biol 19: 473-84]. The forward primer 5′-CCTACCTCGGAAGCCATGGTTGCAC-3′ incorporates a new start codon (bold) and a Nco I restriction site (underlined) for cloning applications. The reverse primer 5′-CATTTTACATTGCTAGCACCAGATTC-3′ contains a Nhe I restriction site (underlined) for downstream sub-cloning purposes. The plasmid pNH8 was used as the template DNA in the PCR (30 cycles) using Pfu polymerase under standard conditions and produced a fragment of the expected size ˜1.3 kb. The resulting thmg1 gene codes for amino acids 153-575 of the full-length (575) hmg1 sequence (FIG. 11b of PCT/EP/00/09374). The thmg1 PCR product was cloned into the PGEM-T vector (Promega) according to the manufacturers' instructions and sequenced to confirm fidelity. The H. brasiliensis tHMG1 was inserted into pNH4 (see PCT/EP/00/009,374 between the Nco I and Nhe I sites of the polylinker, which lie between the CaMV 35S double promoter and nos terminator, giving pMH3 (see PCT/EP/00/09374. This chimaeric gene was isolated by digestion with Xma CI and Sal I, purified and cloned into the corresponding polylinker sites in pNH9, after removal of the chimaeric full length hmg1 gene which previously occupied these sites, and subsequent purification of the binary vector. The binary vector pNH9 also contains the smt1 gene cloned from Nicotiana tabacum, which is under transcriptional control of the CERV viral promoter. This binary construct was named pMH7 (FIG. 2). As described in Example 1, binary vectors were transformed into Agrobacterium tumefaciens and these were subsequently used to transform tobacco.

Table 3 shows the sterol analysis of leaf samples obtained from tobacco transformed with the MH7 vector co-expressing the truncated Hevea HMGR and tobacco sterol methyltransferasel (SMT1). Leaves from 32 independent transgenic plants (MH7) were analysed along with 4 untransformed SR1 controls and 4 vector controls (SJ34). The total sterol content of the SR1 control leaves ranged from 0.141-0.221% dry weight and those of the SJ34 vector control plants from 0.183-0.330%. The total sterol content of the MH7 transgenic plants ranged from 0.142-1.339% dry weight representing increases of up to 7.2-fold over the mean SR1 control value. The beneficial 4-desmethylsterol contents of the MH8 seeds were increased by up to 3.9-fold over the mean SR1 control value.

Table 4 shows the sterol analysis of mature seed samples obtained from tobacco transformed with the MH7 vector co-expressing the truncated Hevea HMGR and tobacco sterol methyltransferasel. Seeds from 29 independent transgenic plants (MH7) were analysed along with 9 SR1 untransformed control plants and 6 vector control plants (SJ34). Seeds from the SR1 control plants show total sterol contents ranging from 0.393-0.445% dry weight and those from the SJ34 vector control plants from 0.334-0.413% dry weight. Seeds from the MH7 plants showed total sterol contents ranging from 0.379-0.987% dry weight, representing increases of up to 2.4-fold over the mean SR1 control value. The beneficial 4-desmethylsterol content of the MH8 seeds was increased by up to 19-fold over the mean SR1 control value. The absolute levels of the 4-desmethylsterols isofucosterol, sitosterol and campesterol were substantially enhanced in the oil control to control values. Percentage cholesterol levels were reduced by up to 73% compared to mean SR1 control values. The increase in ‘beneficial’ 4-desmethylsterols obtained by co-expression of truncated Hevea HMGR and SMT1 is greater than the corresponding increase obtained by expression of the truncated Hevea HMGR alone (see our non-pre-published patent applications PCT/EP00/09374 and EP 00303193.7).

Further analysis of two high sterol seed samples (MH7 53 and MH7 32) was carried out to determine the proportion of free and esterified sterol. The total lipid fraction is isolated as described in Example 2, but not subjected to the transmethylation process. The lipid residue, which contains dihydrocholesterol as internal standard, is dissolved in 40-60 petroleum ether (250 μL) and applied to a glass-backed 20 cm×20 cm×0.5 mm silica gel thin layer chromatography (TLC) plate. The vial that contained the lipid residue is washed out with a further 250 μL aliquot of petroleum ether, which is also applied to the plate. A 10 μL aliquot of a solution consisting of a mixture of β-sitosterol (10 mg) and cholesterol oleate (10 mg) dissolved in acetone (1 mL) is spotted to act as a marker. The plate is developed using 60-80 petroleum ether-diethyl ether-acetic acid (80:20:2, v/v/v). The sterol fractions are visualised by spraying with a 0.01% w/v ethanolic solution of rhodamine 6G and viewing the plate under UV light.

Approximate R_{f} values are 0.25 for free sterols and 0.9 for steryl esters. The free sterol band is scraped off the plate and transferred to a vial. The free sterol fraction is isolated by washing the band with three volumes of diethyl ether. The ether washings are combined and filtered. The free sterol fraction, isolated by blowing off the solvent with nitrogen gas, is silylated and analysed by gas chromatography (GC) as described in Example 1. Amounts of esterified sterol are determined by subtracting amounts of free sterol from total sterol, the latter being determined by transmethylation (see Example 1).

Table 5 shows the analyses of the free sterol and sterol ester fractions of transgenic MH7 seed samples 32 and 53, alongside that of an SR1 control sample. The additional sterol present in the transgenic samples compared to the control is primarily in the form of sterol esters. The total sterol content of the SR1 control is 0.388% dry weight, of which 52.4% is in the form of esters. The total sterol contents of MH7 32 and 53 are 0.965% and 0.987% dry weight respectively, of which 77.2% and 75.0% respectively are esterified.

### EXAMPLE 3

### Co-Expression of a Truncated Form of S. cerevisiae HMGRL and N. tabacum SMT1 in Plants

Saccharomyces cerevisiae NCYC 957, X2180, SUC2 was grown in liquid media (12% (w/v) glucose, 2% (w/v) Bactopeptone, 1% (w/v) yeast extract, pH 4.0) on a rotary shaker (125 rpm), at 30° C. Cells were harvested by centrifuging 50 ml of culture at 4,500 rpm for 10 minutes. To the cell pellet, 4 ml of buffer (50 mM Tris-HCl, pH 8.0, 200 mM NaCl, 100 mM EDTA, 1% SDS) was added and heated at 60° C. for 15 minutes. 40 μl RNase (1 mg/ml) and 40 mg Proteinase K were then added to the mixture prior to heating at 50° C. for 15 minutes. The DNA was extracted twice with phenol/chloroform and once with chloroform. The aqueous layer was added to 0.7 volumes of isopropanol and 3 M sodium acetate, pH 5.2, incubated at room temperature for 1 minute and centrifuged at 13,000 rpm for 10 minutes. The supernatant was removed and 500 μl 70% ethanol was added to the DNA pellet and re-centrifuged. The ethanol was removed and the DNA air dried for 60 minutes. The DNA pellet was suspended in 100 μl TE buffer and the absorbance at 260 nm measured and the DNA quantified. The DNA was diluted to 0.5 μg/μl and frozen.

Based on the nucleotide sequence of cosmid 8248 from the S. cerevisiae chromosome XIII sequencing project, primers were designed to clone the tHMG1 gene by polymerase chain reaction. The forward primer 5′-GCTTGGATAAGG CCATGGGTCCTTTAG-3′ incorporates a new start codon (bold) and a Nco I restriction site (underlined) for cloning purposes. The reverse primer 5′-GAATA CCAATGAGCTCTGACTAAG-3′ contains a Sac I restriction site (underlined) for sub-cloning applications. Prior to PCR the genomic DNA from S. cerevisiae, NCYC 957, X2180, SUC2, mal, gal2, CUA was digested with Eco RI and the DNA fractionated on a 0.7% agarose gel. DNA fragments ˜2.0 kb in size were excised from the gel and purified using the Qiagen QIAquick gel extraction kit, according to the manufacturers protocol. This DNA was used as the template in the subsequent PCR. The PCR (35 cycles) was performed using Tag and Pfu polymerase (3:1) under standard conditions and produced a DNA fragment of the expected size ˜1.4 kb. The resulting tHMGR1 gene codes for amino acids 598-1054 of the full length (1054) HMGR1 sequence (see FIG. 12b of PCT/EP/00/09374). The tHMG1 PCR product was cloned into the pGEM-T vector (Promega) according to the manufacturers' instructions and sequenced to confirm fidelity.

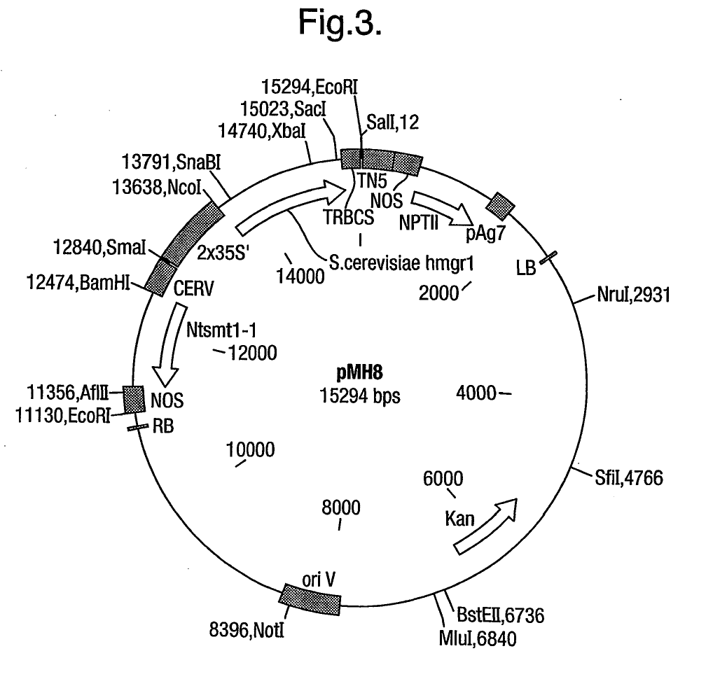

The S. cerevisiae tHMG1 was inserted into pNH4 between the Nco I and Sac I sites of the polylinker pMH4. This chimaeric gene was isolated by digestion with Xma CI and Sal I, purified and cloned into the corresponding polylinker sites in pNH9 as described previously for the H. brasiliensis thmg1 chimaeric gene, to create the binary plasmid pMH8 (FIG. 3). Both pMH3 and pMH4 (see PCT/EP/00/09374) were sequenced to check that the HMG1 genes had been inserted correctly and there were no mistakes in the promoter-initiation and terminator sequences. As described in Example 1, binary vectors were transformed into Agrobacterium tumefaciens and these were subsequently used to transform tobacco.

Table 6 shows the sterol analysis of mature seed samples obtained from tobacco plants transformed with the MH8 vector expressing the truncated S. cerevisiae HMGR and tobacco SMT1 genes. Seeds from 23 independent transgenic plants (MH8) were analysed along with seeds from 4 SR1 control and 4 SJ34 vector control plants. The total sterol content of seeds from the SR1 control plants ranged from 0.363%-0.428% (average=0.388%) and those from the vector control plants from 0.213-0.428%. The total sterol content of the MH8 transgenic seeds ranged from 0.251%-0.526% representing increases of up to 35% over the SR1 average. The 4-desmethylsterol content of the MH8 seeds was increased by up to 41% compared to the SR1 average.

### EXAMPLE 4

### Re-Transformation of ACP—Ntsmt-1 Transgenic Tobacco Plant #27 with an N-Truncated Form of Hevea HMGR Gene Driven by a Constitutive Promoter

Nicotiana tabacum plants (NH19 series) transformed with the N. tabacum Ntsmt-1 gene (SMT1) were generated as described in EP 00303193.7. Seeds from NH19 plant #27 EP 00303193.7 were germinated on MS agar containing 25 mg/L hygromycin. From the resulting seedlings, leaf segments were cut and transformed with a 2×35S—truncated Hevea brasiliensis HMGR construct (MH 5, PCT/EP/00/09374) as described hereabove.

Table 7 shows the sterol analysis of mature seed obtained from NH19 #27 tobacco plants transformed with the MH5 construct and expressing the tobacco SMT1 and truncated H. brasiliensis HMGR genes. Seeds from 24 independent transgenic plants were analysed along with seeds from 5 SR1 control plants, 4 plants grown from NH19 #27 seed and 10 vector control plants (SJ34 into NH19#27). The total sterol content of the SR1 plants ranged from 0.375-0.441% dry weight with an average of 0.413%, those from the NH19#27 plants from 0.413-0.555% dry weight with an average of 0.496% and the vector controls from 0.409%-0.560% dry weight with an average of 0.501%. The total sterol content of the MH5/NH19#27 plants ranged from 0.480-0.928% dry weight representing increases of up to 2.2-fold in total sterols over the SR1 control mean. The 4-desmethylsterol content of the MH5/NH19#27 seeds was increased by up to 1.9-fold over the SR1 average. The increase in ‘beneficial’ 4-desmethylsterols is greater than the corresponding increase in 4-desmethylsterols obtained by expression in tobacco of the truncated HMGR alone (see PCT/EP/00/09374 and EP 00303193).

### EXAMPLE 5

### Re-Transformation of ACP—Ntsmt 1 Transgenic Tobacco Plant 27 with an N-Truncated Hevea HMGR Gene Driven by an 0.29 Kb ACP Seed-Specific Promoter (MH 15)

Nicotiana tabacum plants (NH19 series) transformed with the N. tabacum Ntsmt-1 gene (SMT1) were generated as described in EP 00303193.7. Seeds from NH19 plant #27 EP 00303193.7 were germinated on MS agar containing 25 mg/L hygromycin. From the resulting seedlings, leaf segments were cut and transformed with the Hevea brasiliensis hmg1 gene driven by a 0.29 kb seed-specific Brassica napus acyl carrier protein (ACP) promoter (MH 15 as in PCT/EP/00/09374) as described hereabove.

Table 8 shows the sterol analysis from mature seed from NH19#27 plants re-transformed with MH15 containing the truncated H. brasiliensis hmg1 gene driven by the ACP promoter. Seeds from 30 independent transgenic plants were analysed along with 4 SR1 control plants, 5 NH19#27 plants and 4 vector control plants (SJ34 into NH19#27). The total sterol content of the SR1 plants ranged from 0.340%-0.432% dry weight with an average of 0.393%, those from the NH19#27 plants from 0.505%-0.595% dry weight with an average of 0.565% and those from vector controls from 0.509%-0.573% dry weight with an average of 0.545%. The total sterol content of the MH15/NH19#27 plants ranged from 0.430%-0.865% dry weight representing increases of up to 2.2-fold in total sterols over the SR control average. The ‘beneficial’ 4-desmethylsterol content of the MH1S/NH19#27 plants was increased by up to 2.3-fold over the SR1 control. The expression of both truncated HMGR and SMT1 genes via seed specific ACP promoters has led to a greater fold increase in ‘beneficial’ 4-desmethylsterols than total sterols.

### EXAMPLE 6

### Re-Transformation of ACP—Ntsmt 1 Transgenic Tobacco Plant 27 with an N-Truncated Hevea brasiliensis hmg1 Gene Driven by a 1.4 Kb Seed Specific ACP Promoter (NH61)

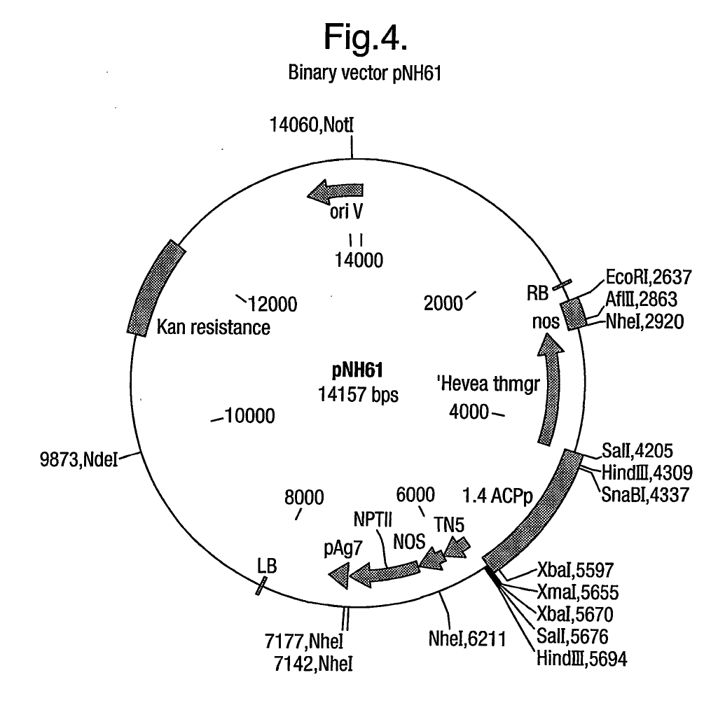

Nicotiana tabacum plants (NH19 series) transformed with the N. tabacum Ntsmt-1 gene (SMT1) were generated as described in EP 00303193.7. Seeds from NH19 plant #27 EP 00303193.7 were germinated on MS agar containing 25 mg/L hygromycin. From the resulting seedlings, leaf segments were cut and transformed with a construct (NH61) containing the N-truncated Hevea brasiliensis HMGR linked to a 1.4 kb seed-specific Brassica napus acyl carrier protein (ACP) promoter. The 1.4 kbp Brassica napus acyl carrier protein (ACP) promoter, including the 5′-untranslated region, was amplified by PCR (primers: clACP1 5′-agg tcg acc cgg gag gat cc-3′, c1ACP2 5′-cag aga gct agc ttg cat gga gac-3′) from vector pTZ5BS [de Silva et al, (1992) Plant Mol Biol 18: 1163-1172], introducing restriction enzyme sites XmaI and NheI (underlined). A truncated version of the Hevea brasiliensis hmgr1 (thmgr1) gene was generated by PCR using vector pHEV36 [Schaller et al., (1995) Plant Physiol 109: 761-770] as the template and primers HbtH1 (5′-acg cGT CGA CTC CCT TAG TCT CGG AGG AAG ACG-3′) and HbtH2 (5′-tcg agc tcc aat tgg cta gc-3′). This gene fragment lacks the 5′-end, which encodes the membrane-spanning domain, and gives rise to a gene product that comprises amino acids 153-575 of the native protein. Restriction enzyme sites SalI and NheI was introduced in either end of the fragment to facilitate cloning. The amplified 1.4 kbp ACP promoter and thmgr1 fragments were digested, ligated and inserted in a modified poly-linker region of pUC19, yielding vector pNH60. The expression cassette, ACP-thmgr1-NOS, was released and cloned into XmaI/EcoRI digested pSJ34 giving binary vector pNH61 (FIG. 4). Binary vector pSJ34 had previously been created by filling in the BamHI site of pGPTV-Kan, between the selectable marker and the p(A)g7 3′-end, with Klenow enzyme [Becker et al., (1992) Plant Mol Biol 20: 1195-97].

Table 9 shows the sterol analysis of mature seed obtained from NH19#27 re-transformed with NH61 and expressing the tobacco SMT1 and the truncated Hevea brasiliensis HMGR. Seeds from 20 independent transgenic plants were analysed along with seeds from 5 SR1 plants and 4 plants grown from NH19#27 seed. The total sterol content of the SR1 seeds ranged from 0.389%-0.459% dry weight with an average of 0.421% and those from NH19#27 T1 plants from 0.489%-0507% dry weight with an average of 0.499%. The total sterol content of seeds from the NH19#27/NH61 plants ranged from 0.497%-1.264% dry weight representing increases of up to 3.0-fold over the SR1 control average. Co-expression of the truncated Hevea HMGR and tobacco SMT1 genes via ACP promoters enhanced total sterols to a greater level than that achieved any other tested combination of the two genes. The 4-desmethylsterol content of the NH19#27/NH61 plants was increased by up to 2.5-fold over the SR1 average. ‘Beneficial’ 4-desmethylsterols as a proportion of total sterols in these transgenic seeds are clearly very high. Levels of sitosterol, campesterol and isofucosterol are particularly elevated, whilst levels of cholesterol are decreased.

### EXAMPLE 7

### Co-Transformation of N. tabacum with a Truncated Form of Hevea brasiliensis HMGR and N. tabacum SMT1 Both Driven by a 1.4 Kb Seed-Specific ACP Promoter

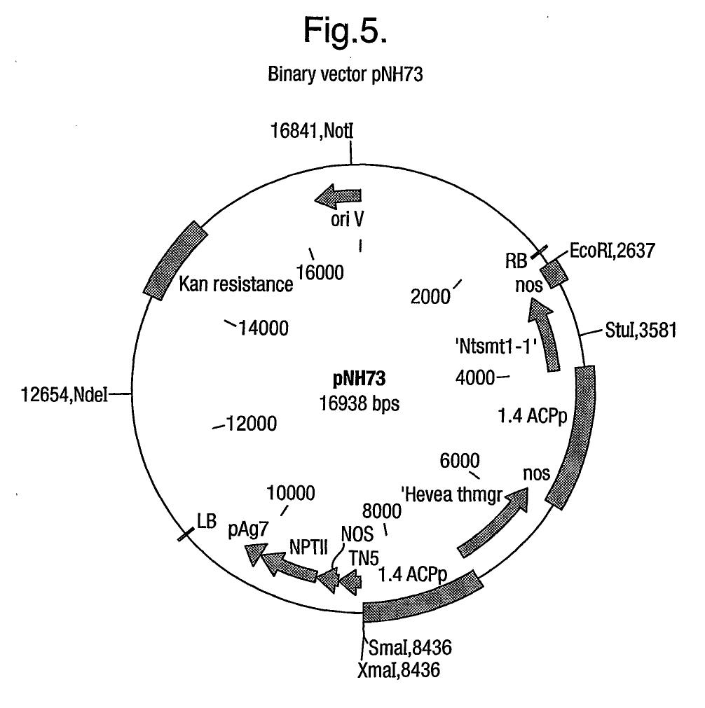

The Ntsmt1-1 gene fragment, encoding Nicotiana tabacum sterol methyltransferase type 1, was amplified by PCR (primers: clSMT1p1 5′-aa cca ATG TCg AcA CAA GGG GCT TTT g-3′, clSMT1p2 5-tcc aat gct agc TTA CTG AGA GTC TGA AAT GG-3′) to introduce SalI and NheI sites (underlined). The amplified Ntsmt1-1 fragment was digested and inserted together with the 1.4 kb Brassica napus ACP promoter fragment (see Example 6) into a modified poly-linker region of pUC19, which also contains the NOS terminator region, yielding vector pNH70. A DNA linker holding an EcoRV site and ends compatible with EcoRI and NdeI was obtained by annealing oligonucleotides EcoV1 (5′-aat tgt atg ata tcg agc tcg aat tcg cgg ccg cca-3′) and EcoV2 (5′-tat ggc ggc cgc gaa ttc gag ctc gat atc ata c-3′). This linker was inserted into the EcoRI/NdeI digested pNH60 yielding pNH71. The SmaI/EcoRI fragment (1.4 ACP promoter-Ntsmt1-1-NOS) was released from pNH70 and inserted into EcoRV/EcoRI digested pNH71 to give pNH72. Vector pNH72 was digested with XmaI and EcoRI to release the double expression cassette (1.4 CP-thmgr1/Ntsmt1-1-NOS), which was subsequently inserted into binary vector pSJ34 to give pNH73 (FIG. 5).

As described in Example 1, pNH73 was transformed into Agrobacterium tumefaciens that, in turn, was used to transform N. tabacum.

### EXAMPLE 8

### Co-Transformation of Brassica napus (Oil Seed Rape) with a Truncated Form of Hevea brasiliensis HMGR and Nicotiana tabacum SMT 1 (MH7)

Electrocompetent Agrobacterium tumefaciens cells (strain LBA4404) were defrosted on ice and 5 ng of pMH7 plasmid (see Example 2) added. Cells plus plasmid were then placed into a pre-chilled electroporation cuvette and electroporated in a Bio Rad Gene Pulser at a capacitance of 25 μF and at 600 ohms. Immediately after electroporation 950 μl of 2×TY broth was added, the cells mixed gently and placed in a sterile vial. The cells were shaken at 28° C. for 2 hours and 25 μl aliquots plated on solid Lennox media containing rifampicin 50 μg/ml and kanamycin 50 μg/ml and incubated at 28° C. for 3 days. Single colonies were used to inoculate 10 μl of water (for PCR confirmation) and 500 μl of Lennox media containing rifampicin 50 μg/ml and kanamycin 50 μg/ml.

Seeds were surface sterilised in 1% sodium hypochlorite for 20 mins. The seeds were washed in sterile distilled water 3 times and plated at a density of 10 seeds per plate on MSMO with 3% sucrose pH 5.8. Seeds were germinated at 24° C. in a 16 h light/8 h dark photoperiod. After 3-4 days, the cotyledons, including 2 mm of petiole, were excised. Care was taken to remove the apical meristem and to keep the cotyledon out of the medium. The excised cotyledons were placed on MS medium, 3% sucrose and 0.7% agar with 20 μM 6-benzylaminopurine (BAP). Petioles with attached cotyledons were embedded in this medium to a depth of approximately 2 mm at 10 per plate.

For transformation, individual excised cotyledons were taken from the plates and the cut surface of their petiole immersed into the agrobacterium suspension for a few seconds. They were then returned to the MS plates and co-cultivated with the agrobacterium for 72 h. After co-cultivation, the cotyledons were transferred to regeneration medium (MS medium with 20M BAP, 3% sucrose, 0.7% agar, pH 5.8 with 400 mg/l augmentin and 15 mg/l kanamycin sulphate). The petioles were, as before, embedded to a depth of 2 mm at a density of 10 explants per plate, and again the cotyledon was kept out of the medium. After 2 or 3 weeks, shoots had appeared, some of which bleached by the fourth week, the remaining green shoots were sub-cultured onto shoot elongation medium (regeneration medium minus BAP). After 1 or 2 weeks, when apical dominance had been established, the shoots were transferred to rooting medium [MS medium, 3% sucrose, 2 mg/l indole butyric acid (IBA), 0.7% agar and 400 mg/l augmentin (no kanamycin)]. As soon as a small root mass was obtained, the plantlets were transferred to potting mix supplemented with fertiliser granules. The plants were grown in a misting chamber (average humidity 75%) for 2-3 weeks at 24° C., 16 h light/8 h dark photoperiod. After 3 weeks the plants were transferred to the glasshouse and allowed to flower and set seed. Mature pods were harvested and seeds subjected to sterol analysis as described in Example 1.

Table 10 shows sterol analysis of mature seed from MH7 transformed plants. Seeds from 4 independent plants were analysed along with seed from a vector control plant. The sterol content of the vector control was 0.243% dry weight, whilst that of the MH7 transgenics ranged from 0.277%-0.374% dry weight representing an increase of up to 1.5-fold in total sterols and 1.6-fold increase in ‘beneficial’ 4-desmethylsterols.

TABLE 1
Sterol Analysis of Leaf from Tobacco transformed with Hevea HMGR + N. tabaccum SMT 1 (NH9)
Total sterols as % of smpl wt

| Smpl | | | | | | | | | | | | |
|---|---|---|---|---|---|---|---|---|---|---|---|---|
| code | squalene | cycloart | 24 mca | 24 mloph | 24 eloph | d7-avena | isofuc | sito | stig | camp | chol | Total |
| NH9 36 | 0.0000 | 0.0061 | 0.0000 | 0.0000 | 0.0066 | 0.0000 | 0.0135 | 0.0700 | 0.1077 | 0.1098 | 0.0041 | 0.318 |
| NH9 37 | 0.0000 | 0.0225 | 0.0000 | 0.0127 | 0.0000 | 0.0000 | 0.0343 | 0.0262 | 0.0811 | 0.0834 | 0.0294 | 0.290 |
| NH9 40 | 0.0000 | 0.0072 | 0.0000 | 0.0055 | 0.0000 | 0.0000 | 0.0197 | 0.0227 | 0.0902 | 0.0955 | 0.0202 | 0.261 |
| NH9 22 | 0.0000 | 0.0000 | 0.0000 | 0.0103 | 0.0000 | 0.0000 | 0.0100 | 0.0466 | 0.0857 | 0.0991 | 0.0000 | 0.252 |
| NH9 11 | 0.0000 | 0.0000 | 0.0000 | 0.0000 | 0.0000 | 0.0000 | 0.0093 | 0.0473 | 0.0963 | 0.0963 | 0.0000 | 0.249 |
| NH9 7 | 0.0000 | 0.0067 | 0.0000 | 0.0113 | 0.0000 | 0.0000 | 0.0229 | 0.0226 | 0.0732 | 0.0842 | 0.0269 | 0.248 |
| NH9 30 | 0.0000 | 0.0000 | 0.0000 | 0.0028 | 0.0000 | 0.0000 | 0.0077 | 0.0410 | 0.0891 | 0.1022 | 0.0033 | 0.246 |
| NH9 28 | 0.0000 | 0.0000 | 0.0000 | 0.0038 | 0.0000 | 0.0000 | 0.0106 | 0.0388 | 0.0891 | 0.0962 | 0.0029 | 0.241 |
| NH9 21 | 0.0000 | 0.0000 | 0.0000 | 0.0024 | 0.0000 | 0.0000 | 0.0101 | 0.0416 | 0.0797 | 0.0871 | 0.0000 | 0.221 |
| NH9 31 | 0.0000 | 0.0000 | 0.0000 | 0.0000 | 0.0000 | 0.0000 | 0.0043 | 0.0380 | 0.0767 | 0.0816 | 0.0000 | 0.201 |
| NH9 18 | 0.0000 | 0.0000 | 0.0000 | 0.0000 | 0.0000 | 0.0000 | 0.0035 | 0.0259 | 0.0694 | 0.0836 | 0.0000 | 0.182 |
| NH9 25 | 0.0000 | 0.0000 | 0.0000 | 0.0000 | 0.0000 | 0.0000 | 0.0025 | 0.0181 | 0.0759 | 0.0793 | 0.0000 | 0.176 |
| SR1 6 | 0.0000 | 0.0088 | 0.0000 | 0.0036 | 0.0000 | 0.0000 | 0.0212 | 0.0303 | 0.0880 | 0.0958 | 0.0204 | 0.268 |
| SR1 7 | 0.0000 | 0.0062 | 0.0000 | 0.0018 | 0.0000 | 0.0000 | 0.0159 | 0.0267 | 0.0831 | 0.0959 | 0.0202 | 0.250 |
| SR1 9 | 0.0000 | 0.0000 | 0.0000 | 0.0057 | 0.0000 | 0.0000 | 0.0244 | 0.0377 | 0.0783 | 0.0828 | 0.0206 | 0.249 |
| SR1 8 | 0.0000 | 0.0031 | 0.0000 | 0.0032 | 0.0000 | 0.0000 | 0.0181 | 0.0259 | 0.0797 | 0.0890 | 0.0189 | 0.238 |
| SR1 10 | 0.0000 | 0.0070 | 0.0000 | 0.0045 | 0.0000 | 0.0000 | 0.0154 | 0.0173 | 0.0802 | 0.0807 | 0.0200 | 0.225 |
| SR1 3 | 0.0000 | 0.0000 | 0.0000 | 0.0000 | 0.0000 | 0.0000 | 0.0129 | 0.0453 | 0.0472 | 0.0487 | 0.0108 | 0.165 |
| Average | 0.0000 | 0.0042 | 0.0000 | 0.0031 | 0.0000 | 0.0000 | 0.0180 | 0.0305 | 0.0761 | 0.0822 | 0.0185 | 0.233 |
| pVEC 1 | 0.0000 | 0.0059 | 0.0000 | 0.0086 | 0.0000 | 0.0000 | 0.0291 | 0.0409 | 0.0760 | 0.0875 | 0.0209 | 0.269 |
| pVEC 18 | 0.0000 | 0.0094 | 0.0027 | 0.0054 | 0.0000 | 0.0000 | 0.0306 | 0.0254 | 0.0766 | 0.0702 | 0.0174 | 0.238 |
| pVEC 5 | 0.0000 | 0.0019 | 0.0000 | 0.0040 | 0.0000 | 0.0000 | 0.0197 | 0.0349 | 0.0710 | 0.0764 | 0.0166 | 0.224 |
| pVEC 17 | 0.0000 | 0.0055 | 0.0000 | 0.0029 | 0.0000 | 0.0000 | 0.0175 | 0.0265 | 0.0710 | 0.0822 | 0.0186 | 0.224 |
| pVEC 10 | 0.0000 | 0.0000 | 0.0000 | 0.0024 | 0.0000 | 0.0000 | 0.0108 | 0.0226 | 0.0653 | 0.0643 | 0.0094 | 0.175 |
| Average | 0.0000 | 0.0045 | 0.0005 | 0.0046 | 0.0000 | 0.0000 | 0.0215 | 0.0301 | 0.0720 | 0.0761 | 0.0166 | 0.226 |

TABLE 2
Sterol Analysis of Seed from Tobacco transformed with Hevea HMGR + N. tabaccum SMT 1 (NH9)
Total sterols as % of smpl wt

| Smpl | | | | | | | | | | | | |
|---|---|---|---|---|---|---|---|---|---|---|---|---|
| code | squalene | cycloart | 24 mca | 24 mloph | 24 eloph | d7-avena | isofuc | sito | stig | camp | chol | Total |
| NH9 34 | 0.0000 | 0.0106 | 0.0078 | 0.0142 | 0.0625 | 0.0046 | 0.0938 | 0.2288 | 0.0377 | 0.0692 | 0.0156 | 0.545 |
| NH9 40 | 0.0000 | 0.0096 | 0.0069 | 0.0126 | 0.0566 | 0.0037 | 0.1095 | 0.2062 | 0.0344 | 0.0714 | 0.0166 | 0.528 |
| NH9 21 | 0.0000 | 0.0115 | 0.0081 | 0.0118 | 0.0505 | 0.0027 | 0.0913 | 0.2074 | 0.0451 | 0.0704 | 0.0218 | 0.521 |
| NH9 19 | 0.0000 | 0.0164 | 0.0068 | 0.0095 | 0.0543 | 0.0030 | 0.0798 | 0.1855 | 0.0382 | 0.0588 | 0.0546 | 0.507 |
| NH9 36 | 0.0000 | 0.0099 | 0.0061 | 0.0098 | 0.0437 | 0.0025 | 0.0868 | 0.2022 | 0.0394 | 0.0615 | 0.0164 | 0.478 |
| NH9 11 | 0.0000 | 0.0163 | 0.0066 | 0.0119 | 0.0521 | 0.0034 | 0.0870 | 0.1840 | 0.0342 | 0.0585 | 0.0217 | 0.476 |
| NH9 22 | 0.0000 | 0.0113 | 0.0063 | 0.0101 | 0.0474 | 0.0031 | 0.0804 | 0.1940 | 0.0396 | 0.0599 | 0.0157 | 0.468 |
| NH9 31 | 0.0000 | 0.0158 | 0.0091 | 0.0112 | 0.0516 | 0.0038 | 0.0849 | 0.1731 | 0.0331 | 0.0559 | 0.0256 | 0.464 |
| NH9 10 | 0.0000 | 0.0163 | 0.0069 | 0.0094 | 0.0494 | 0.0031 | 0.0816 | 0.1819 | 0.0320 | 0.0549 | 0.0167 | 0.452 |
| NH9 33 | 0.0000 | 0.0137 | 0.0074 | 0.0093 | 0.0465 | 0.0031 | 0.0790 | 0.1703 | 0.0397 | 0.0531 | 0.0247 | 0.447 |
| NH9 13 | 0.0000 | 0.0146 | 0.0075 | 0.0089 | 0.0417 | 0.0028 | 0.0722 | 0.1768 | 0.0418 | 0.0593 | 0.0187 | 0.444 |
| NH9 32 | 0.0000 | 0.0114 | 0.0074 | 0.0081 | 0.0400 | 0.0023 | 0.0782 | 0.1681 | 0.0401 | 0.0537 | 0.0244 | 0.434 |
| NH9 27 | 0.0000 | 0.0149 | 0.0063 | 0.0071 | 0.0408 | 0.0025 | 0.0702 | 0.1694 | 0.0361 | 0.0525 | 0.0179 | 0.418 |
| NH9 26 | 0.0000 | 0.0133 | 0.0065 | 0.0000 | 0.0382 | 0.0026 | 0.0630 | 0.1781 | 0.0422 | 0.0571 | 0.0152 | 0.416 |
| NH9 29 | 0.0000 | 0.0087 | 0.0036 | 0.0059 | 0.0307 | 0.0018 | 0.0686 | 0.1835 | 0.0403 | 0.0589 | 0.0136 | 0.416 |
| NH9 25 | 0.0000 | 0.0161 | 0.0079 | 0.0067 | 0.0352 | 0.0031 | 0.0676 | 0.1546 | 0.0383 | 0.0534 | 0.0268 | 0.410 |
| NH9 35 | 0.0000 | 0.0178 | 0.0058 | 0.0046 | 0.0372 | 0.0027 | 0.0719 | 0.1504 | 0.0361 | 0.0497 | 0.0269 | 0.403 |
| NH9 39 | 0.0000 | 0.0247 | 0.0082 | 0.0048 | 0.0323 | 0.0019 | 0.0718 | 0.1460 | 0.0343 | 0.0481 | 0.0264 | 0.398 |
| NH9 6 | 0.0000 | 0.0280 | 0.0102 | 0.0044 | 0.0276 | 0.0013 | 0.0611 | 0.1423 | 0.0411 | 0.0505 | 0.0276 | 0.394 |
| NH9 30 | 0.0000 | 0.0110 | 0.0054 | 0.0085 | 0.0366 | 0.0024 | 0.0711 | 0.1485 | 0.0376 | 0.0539 | 0.0163 | 0.391 |
| NH9 12 | 0.0000 | 0.0107 | 0.0055 | 0.0053 | 0.0325 | 0.0025 | 0.0563 | 0.1658 | 0.0426 | 0.0529 | 0.0143 | 0.388 |
| NH9 37 | 0.0000 | 0.0356 | 0.0095 | 0.0000 | 0.0252 | 0.0019 | 0.0587 | 0.1410 | 0.0366 | 0.0448 | 0.0239 | 0.377 |
| NH9 14 | 0.0000 | 0.0289 | 0.0079 | 0.0045 | 0.0315 | 0.0019 | 0.0608 | 0.1335 | 0.0314 | 0.0421 | 0.0205 | 0.363 |
| NH9 24 | 0.0000 | 0.0297 | 0.0089 | 0.0040 | 0.0207 | 0.0018 | 0.0572 | 0.1302 | 0.0351 | 0.0445 | 0.0297 | 0.362 |
| NH9 18 | 0.0000 | 0.0118 | 0.0054 | 0.0000 | 0.0382 | 0.0026 | 0.0578 | 0.1470 | 0.0370 | 0.0465 | 0.0149 | 0.361 |
| NH9 23 | 0.0000 | 0.0077 | 0.0033 | 0.0039 | 0.0152 | 0.0024 | 0.0363 | 0.1371 | 0.0581 | 0.0559 | 0.0127 | 0.333 |
| NH9 18 | 0.0000 | 0.0076 | 0.0000 | 0.0000 | 0.0169 | 0.0000 | 0.0491 | 0.1238 | 0.0432 | 0.0488 | 0.0173 | 0.307 |
| SR1 18 | 0.0000 | 0.0330 | 0.0086 | 0.0058 | 0.0368 | 0.0020 | 0.0766 | 0.1533 | 0.0325 | 0.0497 | 0.0268 | 0.425 |
| SR1 6 | 0.0000 | 0.0337 | 0.0077 | 0.0057 | 0.0347 | 0.0014 | 0.0684 | 0.1416 | 0.0347 | 0.0497 | 0.0314 | 0.409 |
| SR1 3 | 0.0000 | 0.0306 | 0.0093 | 0.0070 | 0.0349 | 0.0029 | 0.0725 | 0.1427 | 0.0317 | 0.0466 | 0.0290 | 0.407 |
| SR1 17 | 0.0000 | 0.0346 | 0.0088 | 0.0043 | 0.0336 | 0.0025 | 0.0678 | 0.1471 | 0.0305 | 0.0459 | 0.0244 | 0.400 |
| SR1 1 | 0.0000 | 0.0310 | 0.0079 | 0.0053 | 0.0337 | 0.0023 | 0.0654 | 0.1357 | 0.0329 | 0.0449 | 0.0271 | 0.386 |
| SR1 9 | 0.0000 | 0.0292 | 0.0071 | 0.0046 | 0.0332 | 0.0021 | 0.0681 | 0.1391 | 0.0312 | 0.0459 | 0.0235 | 0.384 |
| SR1 7 | 0.0000 | 0.0305 | 0.0093 | 0.0048 | 0.0307 | 0.0025 | 0.0647 | 0.1334 | 0.0365 | 0.0468 | 0.0243 | 0.384 |
| SR1 2 | 0.0000 | 0.0212 | 0.0082 | 0.0040 | 0.0266 | 0.0024 | 0.0627 | 0.1391 | 0.0400 | 0.0489 | 0.0267 | 0.380 |
| SR1 8 | 0.0000 | 0.0334 | 0.0093 | 0.0053 | 0.0350 | 0.0019 | 0.0627 | 0.1261 | 0.0284 | 0.0427 | 0.0274 | 0.372 |
| SR1 4 | 0.0000 | 0.0176 | 0.0062 | 0.0000 | 0.0218 | 0.0012 | 0.0599 | 0.1357 | 0.0367 | 0.0431 | 0.0220 | 0.344 |
| SR1 5 | 0.0000 | 0.0125 | 0.0048 | 0.0000 | 0.0177 | 0.0014 | 0.0547 | 0.1413 | 0.0413 | 0.0442 | 0.0226 | 0.341 |
| SR1 20 | 0.0000 | 0.0246 | 0.0064 | 0.0000 | 0.0232 | 0.0012 | 0.0495 | 0.1346 | 0.0407 | 0.0427 | 0.0160 | 0.339 |
| Average | 0.0000 | 0.0277 | 0.0078 | 0.0039 | 0.0301 | 0.0020 | 0.0644 | 0.1392 | 0.0348 | 0.0459 | 0.0251 | 0.381 |
| pVEC 17 | 0.0000 | 0.0319 | 0.0091 | 0.0062 | 0.0382 | 0.0027 | 0.0735 | 0.1416 | 0.0331 | 0.0465 | 0.0305 | 0.413 |
| pVEC 10 | 0.0000 | 0.0284 | 0.0111 | 0.0062 | 0.0377 | 0.0026 | 0.0663 | 0.1453 | 0.0334 | 0.0474 | 0.0290 | 0.407 |
| pVEC 15 | 0.0000 | 0.0257 | 0.0081 | 0.0052 | 0.0314 | 0.0017 | 0.0717 | 0.1443 | 0.0339 | 0.0489 | 0.0341 | 0.405 |
| pVEC 9 | 0.0000 | 0.0235 | 0.0081 | 0.0071 | 0.0347 | 0.0026 | 0.0751 | 0.1409 | 0.0340 | 0.0462 | 0.0305 | 0.403 |
| pVEC 11 | 0.0000 | 0.0242 | 0.0098 | 0.0064 | 0.0312 | 0.0026 | 0.0641 | 0.1537 | 0.0361 | 0.0429 | 0.0227 | 0.394 |
| pVEC 5 | 0.0000 | 0.0131 | 0.0050 | 0.0000 | 0.0147 | 0.0025 | 0.0373 | 0.1221 | 0.0465 | 0.0425 | 0.0173 | 0.301 |
| Average | 0.0000 | 0.0245 | 0.0085 | 0.0052 | 0.0313 | 0.0025 | 0.0646 | 0.1413 | 0.0362 | 0.0457 | 0.0273 | 0.387 |

TABLE 3
Sterol Analysis of Leaf from Tobacco transformed with truncated Hevea HMGR and Nicotiana SMT1 (MH7)
Total sterols as % of dry weight

| Smpl | | | | | | | | | | | | |
|---|---|---|---|---|---|---|---|---|---|---|---|---|
| code | squalene | cycloart | 24 mca | 24 mloph | 24 eloph | d7-avena | isofuc | sito | stig | camp | chol | Total |
| MH7 53 | 0.1504 | 0.1082 | 0.2321 | 0.1357 | 0.1273 | 0.0229 | 0.1490 | 0.2241 | 0.1079 | 0.0714 | 0.0095 | 1.339 |
| MH7 31 | 0.0327 | 0.4200 | 0.0292 | 0.0444 | 0.0509 | 0.0236 | 0.1148 | 0.1010 | 0.0872 | 0.0424 | 0.0315 | 0.978 |
| MH7 32 | 0.0278 | 0.0930 | 0.1479 | 0.0964 | 0.1132 | 0.0194 | 0.1191 | 0.1716 | 0.0899 | 0.0512 | 0.0152 | 0.945 |
| MH7 3 | 0.0189 | 0.0891 | 0.1544 | 0.0891 | 0.0973 | 0.0212 | 0.1346 | 0.1406 | 0.0829 | 0.0460 | 0.0059 | 0.880 |
| MH7 35 | 0.0282 | 0.0825 | 0.2329 | 0.0794 | 0.0802 | 0.0229 | 0.1012 | 0.1208 | 0.0640 | 0.0383 | 0.0110 | 0.861 |
| MH7 19 | 0.0136 | 0.0838 | 0.1311 | 0.0836 | 0.0953 | 0.0201 | 0.1253 | 0.1458 | 0.0776 | 0.0470 | 0.0042 | 0.827 |
| MH7 54 | 0.0075 | 0.0912 | 0.1083 | 0.0825 | 0.0684 | 0.0179 | 0.1280 | 0.1476 | 0.0862 | 0.0633 | 0.0045 | 0.805 |
| MH7 6 | 0.0096 | 0.1312 | 0.0716 | 0.0531 | 0.0641 | 0.0119 | 0.1127 | 0.1167 | 0.0800 | 0.0504 | 0.0059 | 0.707 |
| MH7 4 | 0.0066 | 0.1528 | 0.0201 | 0.0326 | 0.0275 | 0.0042 | 0.0452 | 0.0857 | 0.1042 | 0.0516 | 0.0290 | 0.559 |
| MH7 46 | 0.0000 | 0.0387 | 0.0156 | 0.0170 | 0.0044 | 0.0024 | 0.0175 | 0.0190 | 0.1044 | 0.0279 | 0.0288 | 0.276 |
| MH7 48 | 0.0014 | 0.0215 | 0.0121 | 0.0179 | 0.0108 | 0.0032 | 0.0145 | 0.0396 | 0.0950 | 0.0481 | 0.0087 | 0.273 |
| MH7 43 | 0.0000 | 0.0187 | 0.0115 | 0.0118 | 0.0119 | 0.0059 | 0.0426 | 0.0563 | 0.0590 | 0.0496 | 0.0053 | 0.273 |
| MH7 45 | 0.0000 | 0.0369 | 0.0154 | 0.0158 | 0.0054 | 0.0024 | 0.0121 | 0.0285 | 0.0968 | 0.0356 | 0.0165 | 0.265 |
| MH7 11 | 0.0000 | 0.0070 | 0.0136 | 0.0133 | 0.0058 | 0.0033 | 0.0256 | 0.0327 | 0.1027 | 0.0429 | 0.0177 | 0.264 |
| MH7 42 | 0.0000 | 0.0293 | 0.0093 | 0.0130 | 0.0059 | 0.0016 | 0.0027 | 0.0465 | 0.0894 | 0.0489 | 0.0071 | 0.254 |
| MH7 24 | 0.0000 | 0.0216 | 0.0122 | 0.0118 | 0.0042 | 0.0028 | 0.0157 | 0.0305 | 0.0945 | 0.0371 | 0.0206 | 0.251 |
| MH7 27 | 0.0000 | 0.0074 | 0.0126 | 0.0104 | 0.0047 | 0.0030 | 0.0163 | 0.0302 | 0.1023 | 0.0390 | 0.0225 | 0.249 |
| MH7 33 | 0.0000 | 0.0345 | 0.0140 | 0.0119 | 0.0053 | 0.0026 | 0.0083 | 0.0280 | 0.0917 | 0.0388 | 0.0120 | 0.247 |
| MH7 37 | 0.0013 | 0.0276 | 0.0131 | 0.0105 | 0.0039 | 0.0029 | 0.0162 | 0.0213 | 0.0853 | 0.0318 | 0.0209 | 0.235 |
| MH7 22 | 0.0000 | 0.0226 | 0.0077 | 0.0081 | 0.0038 | 0.0020 | 0.0125 | 0.0191 | 0.1011 | 0.0354 | 0.0218 | 0.234 |
| MH7 34 | 0.0000 | 0.0182 | 0.0082 | 0.0073 | 0.0048 | 0.0028 | 0.0126 | 0.0328 | 0.0914 | 0.0395 | 0.0115 | 0.229 |
| MH7 26 | 0.0000 | 0.0159 | 0.0111 | 0.0105 | 0.0037 | 0.0027 | 0.0164 | 0.0249 | 0.0873 | 0.0406 | 0.0142 | 0.227 |
| MH7 38 | 0.0000 | 0.0130 | 0.0080 | 0.0066 | 0.0058 | 0.0032 | 0.0169 | 0.0310 | 0.0877 | 0.0381 | 0.0163 | 0.227 |
| MH7 21 | 0.0000 | 0.0122 | 0.0041 | 0.0058 | 0.0066 | 0.0033 | 0.0227 | 0.0396 | 0.0726 | 0.0441 | 0.0051 | 0.216 |
| MH7 47 | 0.0008 | 0.0246 | 0.0090 | 0.0096 | 0.0032 | 0.0015 | 0.0126 | 0.0167 | 0.0855 | 0.0258 | 0.0255 | 0.215 |
| MH7 40 | 0.0000 | 0.0256 | 0.0119 | 0.0097 | 0.0044 | 0.0026 | 0.0123 | 0.0230 | 0.0753 | 0.0325 | 0.0150 | 0.212 |
| MH7 36 | 0.0000 | 0.0131 | 0.0064 | 0.0089 | 0.0062 | 0.0019 | 0.0078 | 0.0324 | 0.0734 | 0.0398 | 0.0039 | 0.194 |
| MH7 7 | 0.0000 | 0.0275 | 0.0075 | 0.0064 | 0.0030 | 0.0020 | 0.0146 | 0.0172 | 0.0585 | 0.0262 | 0.0121 | 0.175 |
| MH7 49 | 0.0000 | 0.0088 | 0.0045 | 0.0050 | 0.0025 | 0.0024 | 0.0201 | 0.0141 | 0.0586 | 0.0298 | 0.0184 | 0.164 |
| MH7 5 | 0.0000 | 0.0038 | 0.0023 | 0.0061 | 0.0036 | 0.0025 | 0.0152 | 0.0216 | 0.0640 | 0.0345 | 0.0067 | 0.160 |
| MH7 23 | 0.0000 | 0.0134 | 0.0055 | 0.0083 | 0.0017 | 0.0019 | 0.0131 | 0.0128 | 0.0609 | 0.0243 | 0.0151 | 0.157 |
| MH7 39 | 0.0000 | 0.0059 | 0.0036 | 0.0058 | 0.0037 | 0.0021 | 0.0117 | 0.0182 | 0.0553 | 0.0276 | 0.0085 | 0.142 |
| SR1 6 | 0.0000 | 0.0166 | 0.0055 | 0.0079 | 0.0039 | 0.0017 | 0.0096 | 0.0216 | 0.0721 | 0.0264 | 0.0132 | 0.179 |
| SR1 7 | 0.0000 | 0.0066 | 0.0029 | 0.0038 | 0.0039 | 0.0023 | 0.0098 | 0.0273 | 0.0966 | 0.0362 | 0.0115 | 0.201 |
| SR1 9 | 0.0000 | 0.0163 | 0.0061 | 0.0074 | 0.0036 | 0.0023 | 0.0175 | 0.0321 | 0.0884 | 0.0320 | 0.0152 | 0.221 |
| SR1 10 | 0.0000 | 0.0044 | 0.0024 | 0.0035 | 0.0036 | 0.0024 | 0.0121 | 0.0187 | 0.0588 | 0.0247 | 0.0108 | 0.141 |
| Average | 0.0000 | 0.0110 | 0.0042 | 0.0057 | 0.0037 | 0.0022 | 0.0122 | 0.0249 | 0.0790 | 0.0298 | 0.0127 | 0.185 |
| SJ34 1 | 0.0000 | 0.0116 | 0.0060 | 0.0100 | 0.0063 | 0.0017 | 0.0123 | 0.0250 | 0.0688 | 0.0289 | 0.0127 | 0.183 |
| SJ34 2 | 0.0014 | 0.0260 | 0.0102 | 0.0129 | 0.0078 | 0.0031 | 0.0241 | 0.0349 | 0.1438 | 0.0398 | 0.0264 | 0.330 |
| SJ34 3 | 0.0000 | 0.0072 | 0.0030 | 0.0054 | 0.0044 | 0.0028 | 0.0190 | 0.0262 | 0.1013 | 0.0315 | 0.0141 | 0.215 |
| SJ34 4 | 0.0000 | 0.0101 | 0.0042 | 0.0044 | 0.0033 | 0.0023 | 0.0119 | 0.0260 | 0.0915 | 0.0305 | 0.0137 | 0.198 |
| Average | 0.0004 | 0.0137 | 0.0058 | 0.0082 | 0.0054 | 0.0025 | 0.0168 | 0.0280 | 0.1013 | 0.0327 | 0.0167 | 0.232 |

TABLE 4
Sterol Analysis of Seed from Tobacco transformed with truncated Hevea HMGR and Nicotiana SMT1 (MH7)
T tal sterols as % of dry weight

| Smpl | | | | | | | | | | | | |
|---|---|---|---|---|---|---|---|---|---|---|---|---|
| code | squalene | cycloart | 24 mca | 24 mloph | 24 eloph | d7-avena | isofuc | sito | stig | camp | chol | Total |
| MH7 53 | 0.0204 | 0.0976 | 0.1707 | 0.0473 | 0.0735 | 0.0242 | 0.1345 | 0.2483 | 0.0482 | 0.0989 | 0.0233 | 0.987 |
| MH7 3 | 0.0168 | 0.1062 | 0.1649 | 0.0625 | 0.0714 | 0.0216 | 0.1254 | 0.2345 | 0.0573 | 0.0940 | 0.0175 | 0.972 |
| MH7 32 | 0.0127 | 0.1004 | 0.1281 | 0.0559 | 0.0783 | 0.0286 | 0.1295 | 0.2484 | 0.0636 | 0.0963 | 0.0232 | 0.965 |
| MH7 35 | 0.0144 | 0.0832 | 0.1349 | 0.0445 | 0.0639 | 0.0185 | 0.1228 | 0.2301 | 0.0515 | 0.0907 | 0.0194 | 0.874 |
| MH7 54 | 0.0139 | 0.0836 | 0.1341 | 0.0401 | 0.0705 | 0.0142 | 0.1191 | 0.2253 | 0.0454 | 0.0786 | 0.0252 | 0.850 |
| MH7 31 | 0.0178 | 0.1770 | 0.0449 | 0.0192 | 0.0476 | 0.0133 | 0.1080 | 0.1854 | 0.0486 | 0.0692 | 0.0245 | 0.755 |
| MH7 7 | 0.0272 | 0.0302 | 0.0100 | 0.0263 | 0.1234 | 0.0104 | 0.1248 | 0.2123 | 0.0337 | 0.0674 | 0.0246 | 0.690 |
| MH7 21 | 0.0162 | 0.0217 | 0.0142 | 0.0262 | 0.1089 | 0.0111 | 0.1203 | 0.2298 | 0.0346 | 0.0712 | 0.0191 | 0.673 |
| MH7 26 | 0.0152 | 0.0179 | 0.0113 | 0.0245 | 0.1115 | 0.0101 | 0.1104 | 0.2414 | 0.0321 | 0.0681 | 0.0163 | 0.659 |
| MH7 23 | 0.0147 | 0.1047 | 0.0248 | 0.0161 | 0.0498 | 0.0079 | 0.0973 | 0.1816 | 0.0413 | 0.0642 | 0.0320 | 0.635 |
| MH7 37 | 0.0114 | 0.0549 | 0.0228 | 0.0237 | 0.0594 | 0.0080 | 0.1020 | 0.2008 | 0.0415 | 0.0757 | 0.0230 | 0.623 |
| MH7 47 | 0.0268 | 0.0539 | 0.0078 | 0.0153 | 0.1037 | 0.0096 | 0.1009 | 0.1930 | 0.0312 | 0.0520 | 0.0263 | 0.621 |
| MH7 40 | 0.0122 | 0.0627 | 0.0224 | 0.0186 | 0.0538 | 0.0065 | 0.0895 | 0.1889 | 0.0378 | 0.0666 | 0.0254 | 0.584 |
| MH7 33 | 0.0143 | 0.0278 | 0.0106 | 0.0193 | 0.0795 | 0.0083 | 0.0958 | 0.1968 | 0.0351 | 0.0651 | 0.0206 | 0.573 |
| MH7 49 | 0.0149 | 0.0646 | 0.0070 | 0.0122 | 0.0598 | 0.0067 | 0.1008 | 0.1931 | 0.0307 | 0.0566 | 0.0269 | 0.573 |
| MH7 5 | 0.0190 | 0.0213 | 0.0076 | 0.0175 | 0.0797 | 0.0079 | 0.0978 | 0.2009 | 0.0317 | 0.0638 | 0.0205 | 0.568 |
| MH7 24 | 0.0098 | 0.0810 | 0.0095 | 0.0135 | 0.0433 | 0.0063 | 0.0844 | 0.1698 | 0.0401 | 0.0620 | 0.0230 | 0.543 |
| MH7 39 | 0.0115 | 0.0463 | 0.0091 | 0.0116 | 0.0545 | 0.0066 | 0.0780 | 0.1847 | 0.0405 | 0.0596 | 0.0217 | 0.524 |
| MH7 45 | 0.0112 | 0.0550 | 0.0150 | 0.0138 | 0.0436 | 0.0050 | 0.0805 | 0.1710 | 0.0362 | 0.0584 | 0.0273 | 0.517 |
| MH7 27 | 0.0124 | 0.0184 | 0.0068 | 0.0142 | 0.0735 | 0.0078 | 0.0871 | 0.1915 | 0.0299 | 0.0555 | 0.0177 | 0.515 |
| MH7 48 | 0.0099 | 0.0496 | 0.0103 | 0.0115 | 0.0447 | 0.0063 | 0.0813 | 0.1692 | 0.0403 | 0.0592 | 0.0274 | 0.510 |
| MH7 22 | 0.0124 | 0.0239 | 0.0078 | 0.0139 | 0.0716 | 0.0065 | 0.0834 | 0.1780 | 0.0320 | 0.0535 | 0.0217 | 0.505 |
| MH7 36 | 0.0087 | 0.0205 | 0.0068 | 0.0098 | 0.0540 | 0.0061 | 0.0718 | 0.1677 | 0.0393 | 0.0576 | 0.0177 | 0.460 |
| MH7 46 | 0.0097 | 0.0341 | 0.0047 | 0.0085 | 0.0453 | 0.0051 | 0.0796 | 0.1565 | 0.0333 | 0.0533 | 0.0265 | 0.457 |
| MH7 4 | 0.0109 | 0.0492 | 0.0128 | 0.0101 | 0.0390 | 0.0048 | 0.0651 | 0.1549 | 0.0369 | 0.0512 | 0.0215 | 0.456 |
| MH7 34 | 0.0053 | 0.0147 | 0.0054 | 0.0040 | 0.0534 | 0.0060 | 0.0667 | 0.1726 | 0.0358 | 0.0526 | 0.0158 | 0.432 |
| MH7 43 | 0.0073 | 0.0369 | 0.0062 | 0.0067 | 0.0397 | 0.0045 | 0.0648 | 0.1464 | 0.0367 | 0.0480 | 0.0240 | 0.421 |
| MH7 11 | 0.0091 | 0.0368 | 0.0037 | 0.0057 | 0.0327 | 0.0044 | 0.0723 | 0.1386 | 0.0362 | 0.0459 | 0.0303 | 0.416 |
| MH7 38 | 0.0066 | 0.0356 | 0.0042 | 0.0051 | 0.0336 | 0.0038 | 0.0535 | 0.1363 | 0.0347 | 0.0449 | 0.0204 | 0.379 |
| SR1 9 | 0.0086 | 0.0458 | 0.0055 | 0.0062 | 0.0430 | 0.0051 | 0.0675 | 0.1501 | 0.0362 | 0.0512 | 0.0255 | 0.445 |
| SR1 4 | 0.0075 | 0.0460 | 0.0042 | 0.0069 | 0.0414 | 0.0047 | 0.0735 | 0.1423 | 0.0342 | 0.0468 | 0.0272 | 0.435 |
| SR1 3 | 0.0087 | 0.0453 | 0.0030 | 0.0063 | 0.0404 | 0.0048 | 0.0673 | 0.1407 | 0.0316 | 0.0469 | 0.0249 | 0.420 |
| SR1 2 | 0.0106 | 0.0400 | 0.0044 | 0.0049 | 0.0396 | 0.0046 | 0.0607 | 0.1485 | 0.0357 | 0.0484 | 0.0214 | 0.419 |
| SR1 8 | 0.0075 | 0.0431 | 0.0053 | 0.0055 | 0.0379 | 0.0049 | 0.0626 | 0.1439 | 0.0347 | 0.0487 | 0.0232 | 0.417 |
| SR1 7 | 0.0079 | 0.0400 | 0.0049 | 0.0055 | 0.0421 | 0.0045 | 0.0628 | 0.1403 | 0.0342 | 0.0460 | 0.0234 | 0.412 |
| SR1 5 | 0.0065 | 0.0349 | 0.0051 | 0.0026 | 0.0385 | 0.0049 | 0.0605 | 0.1437 | 0.0311 | 0.0404 | 0.0258 | 0.394 |
| SR1 10 | 0.0062 | 0.0343 | 0.0051 | 0.0042 | 0.0354 | 0.0052 | 0.0603 | 0.1421 | 0.0328 | 0.0456 | 0.0224 | 0.394 |
| SR1 1 | 0.0081 | 0.0368 | 0.0048 | 0.0048 | 0.0383 | 0.0042 | 0.0620 | 0.1372 | 0.0298 | 0.0410 | 0.0258 | 0.393 |
| Average | 0.0080 | 0.0407 | 0.0047 | 0.0052 | 0.0396 | 0.0048 | 0.0641 | 0.1432 | 0.0334 | 0.0461 | 0.0244 | 0.414 |
| SJ34 8 | 0.0091 | 0.0426 | 0.0044 | 0.0058 | 0.0393 | 0.0045 | 0.0659 | 0.1407 | 0.0307 | 0.0451 | 0.0247 | 0.413 |
| SJ34 5 | 0.0092 | 0.0439 | 0.0040 | 0.0061 | 0.0380 | 0.0044 | 0.0625 | 0.1406 | 0.0344 | 0.0474 | 0.0223 | 0.413 |
| SJ34 3 | 0.0091 | 0.0345 | 0.0048 | 0.0050 | 0.0366 | 0.0041 | 0.0625 | 0.1484 | 0.0357 | 0.0463 | 0.0234 | 0.410 |
| SJ34 2 | 0.0084 | 0.0382 | 0.0048 | 0.0050 | 0.0373 | 0.0041 | 0.0592 | 0.1437 | 0.0333 | 0.0450 | 0.0225 | 0.401 |
| SJ34 6 | 0.0079 | 0.0387 | 0.0045 | 0.0044 | 0.0375 | 0.0046 | 0.0584 | 0.1471 | 0.0353 | 0.0445 | 0.0185 | 0.401 |
| SJ34 4 | 0.0049 | 0.0269 | 0.0027 | 0.0028 | 0.0218 | 0.0036 | 0.0414 | 0.1293 | 0.0415 | 0.0429 | 0.0160 | 0.334 |
| Average | 0.0081 | 0.0375 | 0.0042 | 0.0049 | 0.0351 | 0.0042 | 0.0583 | 0.1416 | 0.0351 | 0.0452 | 0.0212 | 0.395 |

TABLE 5
Analysis of free sterol and sterol ester fractions of MH7 transgenic seed samples

| | | | | | | | | | | | |
|---|---|---|---|---|---|---|---|---|---|---|---|
| Sample / Fraction | cycloart | 24 mca | 24 mloph | 24 eloph | d7-avena | isofuc | sito | stig | camp | chol | Total |

Sterols as % dry wt

| | | | | | | | | | | | |
|---|---|---|---|---|---|---|---|---|---|---|---|
| MH7 32 | | | | | | | | | | | |
| Total sterol (TS) | 0.1004 | 0.1281 | 0.0559 | 0.0783 | 0.0286 | 0.1295 | 0.2484 | 0.0636 | 0.0963 | 0.0232 | 0.965 |
| Free sterol (FS) | 0.0097 | 0.0243 | 0.0047 | 0.0152 | 0.0019 | 0.0239 | 0.0702 | 0.0362 | 0.0261 | 0.0050 | 0.217 |
| Sterol ester (TS-FS) | 0.0906 | 0.1039 | 0.0512 | 0.0631 | 0.0267 | 0.1055 | 0.1782 | 0.0274 | 0.0702 | 0.0182 | 0.748 |
| MH7 53 | | | | | | | | | | | |
| Total sterol (TS) | 0.0976 | 0.1707 | 0.0473 | 0.0735 | 0.0242 | 0.1345 | 0.2483 | 0.0482 | 0.0989 | 0.0233 | 0.987 |
| Free sterol (FS) | 0.0122 | 0.0307 | 0.0064 | 0.0222 | 0.0024 | 0.0300 | 0.0774 | 0.0308 | 0.0253 | 0.0044 | 0.242 |
| Sterol ester (TS-FS) | 0.0854 | 0.1400 | 0.0410 | 0.0513 | 0.0218 | 0.1044 | 0.1709 | 0.0174 | 0.0735 | 0.0189 | 0.745 |
| SR1 control | | | | | | | | | | | |
| Total sterol (TS) | 0.0260 | 0.0161 | 0.0000 | 0.0237 | 0.0017 | 0.0534 | 0.1615 | 0.0366 | 0.0486 | 0.0205 | 0.388 |
| Fr e sterol (FS) | 0.0126 | 0.0032 | 0.0000 | 0.0156 | 0.0000 | 0.0191 | 0.0726 | 0.0314 | 0.0244 | 0.0060 | 0.185 |
| Sterol ester (TS-FS) | 0.0134 | 0.0129 | 0.0000 | 0.0081 | 0.0017 | 0.0343 | 0.0889 | 0.0052 | 0.0241 | 0.0145 | 0.203 |

% FS vs. SE for sterol components

| | | | | | | | | | | | |
|---|---|---|---|---|---|---|---|---|---|---|---|
| MH7 32 | | | | | | | | | | | |
| FS | 9.7 | 18.9 | 8.4 | 19.4 | 6.7 | 18.5 | 28.3 | 56.9 | 27.1 | 21.5 | 22.8 |
| SE | 90.3 | 81.1 | 91.6 | 80.6 | 93.3 | 81.5 | 71.7 | 43.1 | 72.9 | 78.5 | 77.2 |
| MH7 53 | | | | | | | | | | | |
| FS | 12.5 | 18.0 | 13.5 | 30.2 | 9.9 | 22.3 | 31.2 | 63.9 | 25.6 | 18.8 | 25.0 |
| SE | 87.5 | 82.0 | 86.5 | 69.8 | 90.1 | 77.7 | 68.8 | 36.1 | 74.4 | 81.2 | 75.0 |
| SR1 control | | | | | | | | | | | |
| FS | 48.6 | 19.9 | 0.0 | 65.9 | 0.0 | 35.8 | 45.0 | 85.7 | 50.3 | 29.1 | 47.6 |
| SE | 51.4 | 80.1 | 0.0 | 34.1 | 100.0 | 64.2 | 55.0 | 14.3 | 49.7 | 70.9 | 52.4 |

TABLE 6
Sterol Analysis of seed from Tobacco transformed with truncated yeast HMGR + N. tabaccum SMT1 (MH8)
Total sterols as % of dry wt
smpl wt

| Smpl | | | | | | | | | | | | |
|---|---|---|---|---|---|---|---|---|---|---|---|---|
| code | squalene | cycloart | 24 mca | 24 mloph | 24 eloph | d7-avena | isofuc | sito | stig | camp | chol | Total |
| MH8 16 | 0.0143 | 0.0092 | 0.0058 | 0.0070 | 0.0647 | 0.0073 | 0.0864 | 0.2106 | 0.0371 | 0.0689 | 0.0143 | 0.526 |
| MH8 53 | 0.0084 | 0.0111 | 0.0081 | 0.0080 | 0.0605 | 0.0071 | 0.0876 | 0.1931 | 0.0360 | 0.0688 | 0.0162 | 0.505 |
| MH8 46 | 0.0090 | 0.0243 | 0.0060 | 0.0125 | 0.0614 | 0.0065 | 0.0740 | 0.1736 | 0.0368 | 0.0587 | 0.0174 | 0.480 |
| MH8 54 | 0.0089 | 0.0172 | 0.0063 | 0.0103 | 0.0567 | 0.0061 | 0.0780 | 0.1741 | 0.0398 | 0.0630 | 0.0158 | 0.476 |
| MH8 56 | 0.0098 | 0.0187 | 0.0064 | 0.0093 | 0.0542 | 0.0056 | 0.0737 | 0.1783 | 0.0406 | 0.0611 | 0.0161 | 0.474 |
| MH8 38 | 0.0088 | 0.0124 | 0.0077 | 0.0062 | 0.0675 | 0.0072 | 0.0749 | 0.1732 | 0.0406 | 0.0568 | 0.0167 | 0.472 |
| MH8 18 | 0.0083 | 0.0129 | 0.0070 | 0.0053 | 0.0553 | 0.0068 | 0.0719 | 0.1836 | 0.0433 | 0.0599 | 0.0148 | 0.469 |
| MH8 19 | 0.0081 | 0.0173 | 0.0082 | 0.0078 | 0.0533 | 0.0065 | 0.0805 | 0.1702 | 0.0348 | 0.0571 | 0.0188 | 0.462 |
| MH8 32 | 0.0052 | 0.0098 | 0.0066 | 0.0055 | 0.0466 | 0.0070 | 0.0883 | 0.1699 | 0.0348 | 0.0601 | 0.0192 | 0.453 |
| MH8 48 | 0.0072 | 0.0453 | 0.0056 | 0.0053 | 0.0334 | 0.0046 | 0.0638 | 0.1459 | 0.0355 | 0.0487 | 0.0260 | 0.421 |
| MH8 14 | 0.0044 | 0.0284 | 0.0075 | 0.0047 | 0.0330 | 0.0058 | 0.0670 | 0.1504 | 0.0349 | 0.0454 | 0.0264 | 0.408 |
| MH8 44 | 0.0065 | 0.0300 | 0.0061 | 0.0060 | 0.0328 | 0.0070 | 0.0688 | 0.1385 | 0.0348 | 0.0449 | 0.0271 | 0.403 |
| MH8 40 | 0.0041 | 0.0056 | 0.0046 | 0.0037 | 0.0396 | 0.0057 | 0.0551 | 0.1608 | 0.0469 | 0.0586 | 0.0099 | 0.395 |
| MH8 12 | 0.0042 | 0.0130 | 0.0048 | 0.0025 | 0.0398 | 0.0054 | 0.0500 | 0.1538 | 0.0456 | 0.0508 | 0.0163 | 0.386 |
| MH8 43 | 0.0060 | 0.0340 | 0.0041 | 0.0049 | 0.0318 | 0.0041 | 0.0626 | 0.1346 | 0.0307 | 0.0430 | 0.0243 | 0.380 |
| MH8 15 | 0.0044 | 0.0244 | 0.0058 | 0.0038 | 0.0282 | 0.0050 | 0.0516 | 0.1403 | 0.0414 | 0.0446 | 0.0220 | 0.371 |
| MH8 11 | 0.0030 | 0.0075 | 0.0058 | 0.0018 | 0.0299 | 0.0045 | 0.0446 | 0.1510 | 0.0556 | 0.0520 | 0.0116 | 0.367 |
| MH8 23 | 0.0030 | 0.0055 | 0.0061 | 0.0024 | 0.0376 | 0.0051 | 0.0491 | 0.1500 | 0.0436 | 0.0523 | 0.0104 | 0.365 |
| MH8 34 | 0.0054 | 0.0299 | 0.0039 | 0.0049 | 0.0318 | 0.0040 | 0.0511 | 0.1322 | 0.0381 | 0.0455 | 0.0176 | 0.364 |
| MH8 29 | 0.0024 | 0.0086 | 0.0054 | 0.0039 | 0.0280 | 0.0041 | 0.0497 | 0.1443 | 0.0468 | 0.0525 | 0.0145 | 0.360 |
| MH8 33 | 0.0027 | 0.0187 | 0.0037 | 0.0025 | 0.0195 | 0.0040 | 0.0377 | 0.1175 | 0.0501 | 0.0474 | 0.0140 | 0.318 |
| MH8 7 | 0.0000 | 0.0036 | 0.0047 | 0.0014 | 0.0098 | 0.0025 | 0.0208 | 0.0990 | 0.0601 | 0.0487 | 0.0068 | 0.257 |
| MH8 8 | 0.0000 | 0.0020 | 0.0050 | 0.0000 | 0.0068 | 0.0019 | 0.0208 | 0.0982 | 0.0604 | 0.0492 | 0.0068 | 0.251 |
| SR1 3 | 0.0085 | 0.0403 | 0.0058 | 0.0059 | 0.0385 | 0.0051 | 0.0589 | 0.1511 | 0.0461 | 0.0505 | 0.0188 | 0.429 |
| SR1 2 | 0.0063 | 0.0287 | 0.0052 | 0.0040 | 0.0311 | 0.0041 | 0.0511 | 0.1448 | 0.0480 | 0.0493 | 0.0161 | 0.389 |
| SR1 4 | 0.0038 | 0.0252 | 0.0051 | 0.0032 | 0.0232 | 0.0043 | 0.0516 | 0.1361 | 0.0466 | 0.0506 | 0.0213 | 0.371 |
| SR1 1 | 0.0065 | 0.0312 | 0.0046 | 0.0046 | 0.0306 | 0.0039 | 0.0514 | 0.1321 | 0.0367 | 0.0432 | 0.0179 | 0.363 |
| Average | 0.0062 | 0.0313 | 0.0052 | 0.0044 | 0.0308 | 0.0043 | 0.0533 | 0.1410 | 0.0443 | 0.0484 | 0.0185 | 0.388 |
| SJ34 13 | 0.0064 | 0.0335 | 0.0057 | 0.0056 | 0.0323 | 0.0067 | 0.0758 | 0.1482 | 0.0332 | 0.0476 | 0.0326 | 0.428 |
| SJ34 11 | 0.0078 | 0.0282 | 0.0055 | 0.0053 | 0.0341 | 0.0048 | 0.0724 | 0.1430 | 0.0359 | 0.0470 | 0.0300 | 0.414 |
| SJ34 18 | 0.0017 | 0.0143 | 0.0047 | 0.0020 | 0.0165 | 0.0034 | 0.0315 | 0.1068 | 0.0515 | 0.0447 | 0.0134 | 0.291 |
| SJ34 17 | 0.0000 | 0.0038 | 0.0027 | 0.0000 | 0.0041 | 0.0021 | 0.0143 | 0.0714 | 0.0632 | 0.0431 | 0.0084 | 0.213 |

TABLE 7
Sterol Analysis of Mature Seed from ACP - NtSmt-1 Tobacco plant #27 re-transformed with N-truncated Hevea
HMGR (MH5)
Total sterols as % of dry weight

| Smpl | | | | | | | | | | | | |
|---|---|---|---|---|---|---|---|---|---|---|---|---|
| code | squalene | cycloart | 24 mca | 24 mloph | 24 eloph | d7-avena | isofuc | sito | stig | camp | chol | Total |
| MH5/27 41 | 0.0168 | 0.1735 | 0.0595 | 0.0295 | 0.0589 | 0.0166 | 0.1573 | 0.2459 | 0.0446 | 0.0997 | 0.0259 | 0.928 |
| MH5/27 11 | 0.0117 | 0.1647 | 0.0532 | 0.0233 | 0.0541 | 0.0125 | 0.1591 | 0.2332 | 0.0446 | 0.0839 | 0.0256 | 0.866 |
| MH5/27 25 | 0.0096 | 0.1257 | 0.0533 | 0.0256 | 0.0626 | 0.0165 | 0.1424 | 0.2343 | 0.0405 | 0.0862 | 0.0205 | 0.817 |
| MH5/27 60 | 0.0132 | 0.1150 | 0.0440 | 0.0254 | 0.0660 | 0.0168 | 0.1403 | 0.2414 | 0.0381 | 0.0806 | 0.0229 | 0.804 |
| MH5/27 2 | 0.0138 | 0.1037 | 0.0405 | 0.0245 | 0.0651 | 0.0136 | 0.1544 | 0.2381 | 0.0385 | 0.0857 | 0.0224 | 0.800 |
| MH5/27 17 | 0.0114 | 0.1147 | 0.0435 | 0.0251 | 0.0537 | 0.0199 | 0.1303 | 0.2267 | 0.0436 | 0.0778 | 0.0229 | 0.769 |
| MH5/27 44 | 0.0113 | 0.1178 | 0.0450 | 0.0256 | 0.0573 | 0.0134 | 0.1404 | 0.2116 | 0.0344 | 0.0743 | 0.0224 | 0.753 |
| MH5/27 31 | 0.0067 | 0.0972 | 0.0435 | 0.0243 | 0.0543 | 0.0176 | 0.1316 | 0.2297 | 0.0419 | 0.0799 | 0.0177 | 0.744 |
| MH5/27 27 | 0.0131 | 0.0736 | 0.0306 | 0.0239 | 0.0598 | 0.0164 | 0.1291 | 0.2350 | 0.0379 | 0.0804 | 0.0211 | 0.721 |
| MH5/27 58 | 0.0131 | 0.0689 | 0.0370 | 0.0236 | 0.0595 | 0.0065 | 0.1253 | 0.2321 | 0.0397 | 0.0836 | 0.0212 | 0.710 |
| MH5/27 39 | 0.0179 | 0.0485 | 0.0108 | 0.0206 | 0.0899 | 0.0114 | 0.1213 | 0.2539 | 0.0374 | 0.0762 | 0.0170 | 0.705 |
| MH5/27 42 | 0.0056 | 0.0805 | 0.0382 | 0.0223 | 0.0477 | 0.0091 | 0.1208 | 0.2187 | 0.0444 | 0.0791 | 0.0188 | 0.685 |
| MH5/27 10 | 0.0117 | 0.0665 | 0.0339 | 0.0179 | 0.0475 | 0.0104 | 0.1046 | 0.1959 | 0.0322 | 0.0662 | 0.0231 | 0.610 |
| MH5/27 28 | 0.0099 | 0.0359 | 0.0100 | 0.0127 | 0.0623 | 0.0092 | 0.1047 | 0.2270 | 0.0360 | 0.0680 | 0.0175 | 0.593 |
| MH5/27 53 | 0.0113 | 0.0404 | 0.0097 | 0.0156 | 0.0616 | 0.0043 | 0.1060 | 0.2174 | 0.0346 | 0.0677 | 0.0181 | 0.587 |
| MH5/27 55 | 0.0098 | 0.0305 | 0.0050 | 0.0133 | 0.0543 | 0.0022 | 0.1063 | 0.2120 | 0.0372 | 0.0779 | 0.0159 | 0.564 |
| MH5/27 57 | 0.0081 | 0.0305 | 0.0048 | 0.0112 | 0.0559 | 0.0029 | 0.0992 | 0.2093 | 0.0341 | 0.0710 | 0.0173 | 0.544 |
| MH5/27 38 | 0.0098 | 0.0323 | 0.0049 | 0.0131 | 0.0501 | 0.0019 | 0.0935 | 0.2069 | 0.0356 | 0.0688 | 0.0168 | 0.534 |
| MH5/27 3 | 0.0095 | 0.0247 | 0.0057 | 0.0093 | 0.0517 | 0.0041 | 0.0924 | 0.2072 | 0.0336 | 0.0652 | 0.0153 | 0.519 |
| MH5/27 48 | 0.0094 | 0.0323 | 0.0039 | 0.0063 | 0.0557 | 0.0024 | 0.0891 | 0.1979 | 0.0353 | 0.0657 | 0.0183 | 0.516 |
| MH5/27 30 | 0.0080 | 0.0241 | 0.0051 | 0.0068 | 0.0455 | 0.0076 | 0.0926 | 0.1957 | 0.0331 | 0.0687 | 0.0164 | 0.504 |
| MH5/27 40 | 0.0110 | 0.0237 | 0.0042 | 0.0061 | 0.0499 | 0.0087 | 0.0886 | 0.1985 | 0.0323 | 0.0624 | 0.0176 | 0.503 |
| MH5/27 7 | 0.0074 | 0.0220 | 0.0038 | 0.0095 | 0.0406 | 0.0033 | 0.0951 | 0.1862 | 0.0365 | 0.0626 | 0.0177 | 0.484 |
| MH5/27 5 | 0.0080 | 0.0269 | 0.0047 | 0.0054 | 0.0470 | 0.0019 | 0.0805 | 0.1930 | 0.0355 | 0.0606 | 0.0171 | 0.480 |
| SJ34/27 15 | 0.0094 | 0.0174 | 0.0033 | 0.0074 | 0.0588 | 0.0018 | 0.1087 | 0.2200 | 0.0347 | 0.0834 | 0.0152 | 0.560 |
| SJ34/27 11 | 0.0082 | 0.0171 | 0.0041 | 0.0066 | 0.0582 | 0.0029 | 0.0989 | 0.2186 | 0.0348 | 0.0772 | 0.0140 | 0.541 |
| SJ34/27 4 | 0.0086 | 0.0181 | 0.0033 | 0.0122 | 0.0559 | 0.0024 | 0.1010 | 0.2107 | 0.0339 | 0.0778 | 0.0140 | 0.538 |
| SJ34/27 14 | 0.0098 | 0.0170 | 0.0034 | 0.0056 | 0.0532 | 0.0017 | 0.1045 | 0.2134 | 0.0327 | 0.0727 | 0.0141 | 0.528 |
| SJ34/27 6 | 0.0088 | 0.0150 | 0.0034 | 0.0060 | 0.0511 | 0.0020 | 0.0976 | 0.2037 | 0.0306 | 0.0724 | 0.0146 | 0.505 |
| SJ34/27 12 | 0.0085 | 0.0211 | 0.0041 | 0.0053 | 0.0526 | 0.0021 | 0.0924 | 0.2027 | 0.0317 | 0.0670 | 0.0146 | 0.502 |
| SJ34/27 13 | 0.0084 | 0.0237 | 0.0029 | 0.0057 | 0.0474 | 0.0032 | 0.0897 | 0.1876 | 0.0366 | 0.0685 | 0.0200 | 0.494 |
| SJ34/27 2 | 0.0079 | 0.0249 | 0.0027 | 0.0056 | 0.0437 | 0.0027 | 0.0890 | 0.1749 | 0.0331 | 0.0600 | 0.0231 | 0.468 |
| SJ34/27 9 | 0.0078 | 0.0191 | 0.0039 | 0.0053 | 0.0416 | 0.0028 | 0.0909 | 0.1790 | 0.0323 | 0.0635 | 0.0189 | 0.465 |
| SJ34/27 8 | 0.0057 | 0.0154 | 0.0030 | 0.0048 | 0.0367 | 0.0013 | 0.0769 | 0.1586 | 0.0310 | 0.0567 | 0.0187 | 0.409 |
| NH19/27 2 | 0.0104 | 0.0195 | 0.0046 | 0.0125 | 0.0580 | 0.0016 | 0.1085 | 0.2109 | 0.0345 | 0.0785 | 0.0156 | 0.555 |
| NH19/27 1 | 0.0095 | 0.0256 | 0.0041 | 0.0118 | 0.0564 | 0.0017 | 0.1055 | 0.2085 | 0.0346 | 0.0721 | 0.0168 | 0.547 |
| NH19/27 4 | 0.0093 | 0.0352 | 0.0029 | 0.0098 | 0.0427 | 0.0022 | 0.0835 | 0.1711 | 0.0339 | 0.0569 | 0.0223 | 0.470 |
| NH19/27 5 | 0.0058 | 0.0114 | 0.0021 | 0.0036 | 0.0285 | 0.0015 | 0.0641 | 0.1776 | 0.0429 | 0.0629 | 0.0127 | 0.413 |
| SR1 3 | 0.0075 | 0.0383 | 0.0035 | 0.0089 | 0.0403 | 0.0022 | 0.0726 | 0.1554 | 0.0360 | 0.0513 | 0.0246 | 0.441 |
| SR1 5 | 0.0083 | 0.0322 | 0.0037 | 0.0084 | 0.0379 | 0.0029 | 0.0714 | 0.1643 | 0.0351 | 0.0516 | 0.0218 | 0.438 |
| SR1 4 | 0.0078 | 0.0422 | 0.0039 | 0.0086 | 0.0409 | 0.0017 | 0.0724 | 0.1534 | 0.0331 | 0.0474 | 0.0240 | 0.435 |
| SR1 2 | 0.0086 | 0.0252 | 0.0042 | 0.0063 | 0.0341 | 0.0050 | 0.0652 | 0.1352 | 0.0295 | 0.0423 | 0.0240 | 0.380 |
| SR1 1 | 0.0072 | 0.0259 | 0.0038 | 0.0037 | 0.0364 | 0.0056 | 0.0614 | 0.1380 | 0.0300 | 0.0405 | 0.0224 | 0.375 |
| Average | 0.0079 | 0.0327 | 0.0038 | 0.0072 | 0.0379 | 0.0035 | 0.0686 | 0.1493 | 0.0327 | 0.0466 | 0.0234 | 0.414 |

Standard deviation for SR1 total sterol = 0.030

TABLE 8
Sterol Analysis of Mature seed from ACP - NtSmt-1 Tobacco plant #27 re-transformed with ACP-N-truncated
Hevea HMGR (MH15)
Total sterols as % of dry weight

| Smpl | | | | | | | | | | | | |
|---|---|---|---|---|---|---|---|---|---|---|---|---|
| code | squalene | cycloart | 24 mca | 24 mloph | 24 eloph | d7-avena | isofuc | sito | stig | camp | chol | Total |
| MH15/27 39 | 0.0099 | 0.0531 | 0.0088 | 0.0249 | 0.0862 | 0.0040 | 0.1768 | 0.3299 | 0.0437 | 0.1113 | 0.0164 | 0.865 |
| MH15/27 28 | 0.0112 | 0.0358 | 0.0079 | 0.0166 | 0.1025 | 0.0038 | 0.1545 | 0.3200 | 0.0377 | 0.0914 | 0.0153 | 0.797 |
| MH15/27 8 | 0.0100 | 0.0394 | 0.0064 | 0.0152 | 0.1092 | 0.0057 | 0.1494 | 0.3036 | 0.0369 | 0.0838 | 0.0196 | 0.779 |
| MH15/27 38 | 0.0097 | 0.0400 | 0.0059 | 0.0180 | 0.0979 | 0.0028 | 0.1482 | 0.2868 | 0.0340 | 0.0902 | 0.0182 | 0.752 |
| MH15/27 3 | 0.0084 | 0.0327 | 0.0065 | 0.0167 | 0.0870 | 0.0029 | 0.1401 | 0.3066 | 0.0381 | 0.0940 | 0.0157 | 0.749 |
| MH15/27 21 | 0.0088 | 0.0209 | 0.0060 | 0.0180 | 0.0706 | 0.0020 | 0.1262 | 0.2781 | 0.0428 | 0.1059 | 0.0134 | 0.693 |
| MH15/27 34 | 0.0095 | 0.0261 | 0.0051 | 0.0169 | 0.0731 | 0.0020 | 0.1418 | 0.2681 | 0.0405 | 0.0911 | 0.0144 | 0.689 |
| MH15/27 15 | 0.0087 | 0.0228 | 0.0056 | 0.0147 | 0.0717 | 0.0028 | 0.1343 | 0.2766 | 0.0404 | 0.0943 | 0.0160 | 0.688 |
| MH15/27 51 | 0.0086 | 0.0285 | 0.0066 | 0.0169 | 0.0734 | 0.0023 | 0.1441 | 0.2660 | 0.0362 | 0.0889 | 0.0164 | 0.688 |
| MH15/27 23 | 0.0093 | 0.0272 | 0.0060 | 0.0145 | 0.0708 | 0.0034 | 0.1263 | 0.2739 | 0.0380 | 0.0901 | 0.0154 | 0.675 |
| MH15/27 9 | 0.0102 | 0.0272 | 0.0046 | 0.0166 | 0.0763 | 0.0020 | 0.1293 | 0.2649 | 0.0353 | 0.0869 | 0.0164 | 0.670 |
| MH15/27 53 | 0.0094 | 0.0242 | 0.0048 | 0.0140 | 0.0681 | 0.0033 | 0.1390 | 0.2635 | 0.0359 | 0.0810 | 0.0172 | 0.660 |
| MH15/27 12 | 0.0082 | 0.0245 | 0.0044 | 0.0134 | 0.0681 | 0.0021 | 0.1271 | 0.2615 | 0.0414 | 0.0903 | 0.0175 | 0.658 |
| MH15/27 59 | 0.0096 | 0.0274 | 0.0057 | 0.0134 | 0.0684 | 0.0031 | 0.1092 | 0.2751 | 0.0428 | 0.0875 | 0.0146 | 0.657 |
| MH15/27 47 | 0.0083 | 0.0279 | 0.0056 | 0.0135 | 0.0669 | 0.0027 | 0.1187 | 0.2664 | 0.0360 | 0.0843 | 0.0145 | 0.645 |
| MH15/27 43 | 0.0087 | 0.0251 | 0.0051 | 0.0123 | 0.0667 | 0.0028 | 0.1230 | 0.2562 | 0.0396 | 0.0797 | 0.0147 | 0.634 |
| MH15/27 42 | 0.0100 | 0.0212 | 0.0039 | 0.0129 | 0.0587 | 0.0025 | 0.1249 | 0.2690 | 0.0392 | 0.0787 | 0.0127 | 0.634 |
| MH15/27 22 | 0.0094 | 0.0280 | 0.0045 | 0.0138 | 0.0672 | 0.0030 | 0.1137 | 0.2593 | 0.0355 | 0.0824 | 0.0151 | 0.632 |
| MH15/27 29 | 0.0075 | 0.0170 | 0.0033 | 0.0114 | 0.0649 | 0.0030 | 0.1027 | 0.2801 | 0.0449 | 0.0818 | 0.0127 | 0.629 |
| MH15/27 4 | 0.0080 | 0.0227 | 0.0048 | 0.0139 | 0.0674 | 0.0032 | 0.1242 | 0.2342 | 0.0353 | 0.0886 | 0.0141 | 0.617 |
| MH15/27 16 | 0.0082 | 0.0236 | 0.0044 | 0.0134 | 0.0666 | 0.0016 | 0.1320 | 0.2197 | 0.0373 | 0.0865 | 0.0157 | 0.609 |
| MH15/27 14 | 0.0090 | 0.0275 | 0.0051 | 0.0137 | 0.0686 | 0.0024 | 0.1100 | 0.2274 | 0.0391 | 0.0814 | 0.0162 | 0.600 |
| MH15/27 17 | 0.0084 | 0.0221 | 0.0047 | 0.0134 | 0.0620 | 0.0021 | 0.1146 | 0.2205 | 0.0371 | 0.0844 | 0.0158 | 0.585 |
| MH15/27 20 | 0.0062 | 0.0217 | 0.0038 | 0.0116 | 0.0419 | 0.0023 | 0.1289 | 0.2357 | 0.0350 | 0.0785 | 0.0131 | 0.579 |
| MH15/27 33 | 0.0065 | 0.0182 | 0.0027 | 0.0106 | 0.0532 | 0.0016 | 0.0908 | 0.2132 | 0.0450 | 0.0766 | 0.0134 | 0.532 |
| MH15/27 31 | 0.0059 | 0.0173 | 0.0044 | 0.0094 | 0.0464 | 0.0032 | 0.1085 | 0.2091 | 0.0353 | 0.0747 | 0.0130 | 0.527 |
| MH15/27 41 | 0.0081 | 0.0168 | 0.0029 | 0.0100 | 0.0525 | 0.0066 | 0.0861 | 0.2123 | 0.0463 | 0.0693 | 0.0147 | 0.526 |
| MH15/27 10 | 0.0061 | 0.0302 | 0.0035 | 0.0089 | 0.0402 | 0.0071 | 0.0656 | 0.1697 | 0.0404 | 0.0526 | 0.0211 | 0.445 |
| MH15/27 40 | 0.0072 | 0.0309 | 0.0036 | 0.0086 | 0.0393 | 0.0022 | 0.0656 | 0.1675 | 0.0374 | 0.0520 | 0.0189 | 0.433 |
| MH15/27 56 | 0.0077 | 0.0261 | 0.0029 | 0.0087 | 0.0366 | 0.0047 | 0.0675 | 0.1716 | 0.0353 | 0.0507 | 0.0184 | 0.430 |
| NH19/27 4 | 0.0080 | 0.0197 | 0.0063 | 0.0126 | 0.0645 | 0.0024 | 0.1226 | 0.2230 | 0.0364 | 0.0863 | 0.0136 | 0.595 |
| NH19/27 2 | 0.0081 | 0.0209 | 0.0063 | 0.0143 | 0.0663 | 0.0033 | 0.1056 | 0.2249 | 0.0369 | 0.0884 | 0.0128 | 0.588 |
| NH19/27 3 | 0.0079 | 0.0161 | 0.0074 | 0.0114 | 0.0633 | 0.0025 | 0.1126 | 0.2273 | 0.0391 | 0.0840 | 0.0119 | 0.584 |
| NH19/27 5 | 0.0089 | 0.0232 | 0.0066 | 0.0143 | 0.0569 | 0.0020 | 0.1045 | 0.2114 | 0.0329 | 0.0791 | 0.0135 | 0.553 |
| NH19/27 1 | 0.0086 | 0.0221 | 0.0048 | 0.0123 | 0.0490 | 0.0020 | 0.1000 | 0.1895 | 0.0308 | 0.0713 | 0.0143 | 0.505 |
| SJ34/27 1 | 0.0095 | 0.0224 | 0.0052 | 0.0157 | 0.0603 | 0.0025 | 0.1088 | 0.2161 | 0.0341 | 0.0827 | 0.0156 | 0.573 |
| SJ34/27 9 | 0.0090 | 0.0227 | 0.0039 | 0.0129 | 0.0594 | 0.0014 | 0.1035 | 0.2136 | 0.0339 | 0.0758 | 0.0152 | 0.551 |
| SJ34/27 13 | 0.0078 | 0.0191 | 0.0058 | 0.0115 | 0.0565 | 0.0024 | 0.1132 | 0.2004 | 0.0351 | 0.0789 | 0.0143 | 0.545 |
| SJ34/27 11 | 0.0086 | 0.0178 | 0.0031 | 0.0099 | 0.0507 | 0.0026 | 0.0878 | 0.2070 | 0.0391 | 0.0697 | 0.0130 | 0.509 |
| SR1 4 | 0.0069 | 0.0320 | 0.0018 | 0.0097 | 0.0373 | 0.0029 | 0.0699 | 0.1561 | 0.0406 | 0.0542 | 0.0208 | 0.432 |
| SR1 5 | 0.0073 | 0.0346 | 0.0037 | 0.0087 | 0.0424 | 0.0036 | 0.0690 | 0.1507 | 0.0307 | 0.0463 | 0.0232 | 0.420 |
| SR1 1 | 0.0055 | 0.0237 | 0.0028 | 0.0063 | 0.0327 | 0.0035 | 0.0551 | 0.1443 | 0.0342 | 0.0441 | 0.0185 | 0.371 |
| SR1 2 | 0.0071 | 0.0206 | 0.0031 | 0.0031 | 0.0323 | 0.0031 | 0.0516 | 0.1381 | 0.0323 | 0.0408 | 0.0173 | 0.349 |
| Average | 0.0067 | 0.0277 | 0.0029 | 0.0069 | 0.0362 | 0.0033 | 0.0614 | 0.1473 | 0.0345 | 0.0463 | 0.0200 | 0.393 |

Standard deviation for SR1 total sterol = 0.034

TABLE 9
Sterol Analysis of Mature seed from ACP - NtSmt-1 Tobacco plant #27 re-transformed with 1.4 kb ACP-Hevea
t-HMGR [NH61])
Total sterols as % of dry weight

| Smpl | | | | | | | | | | | | |
|---|---|---|---|---|---|---|---|---|---|---|---|---|
| code | squalene | cycloart | 24 mca | 24 mloph | 24 eloph | d7-avena | isofuc | sito | stig | camp | chol | Total |
| NH61/27 11 | 0.0475 | 0.1054 | 0.0203 | 0.0689 | 0.2642 | 0.0245 | 0.2123 | 0.3423 | 0.0446 | 0.1186 | 0.0151 | 1.264 |
| NH61/27 16 | 0.0517 | 0.1009 | 0.0154 | 0.0555 | 0.2246 | 0.0206 | 0.2036 | 0.3287 | 0.0423 | 0.1086 | 0.0184 | 1.170 |
| NH61/27 12 | 0.0537 | 0.0968 | 0.0175 | 0.0572 | 0.2204 | 0.0216 | 0.2010 | 0.3310 | 0.0448 | 0.1099 | 0.0152 | 1.169 |
| NH61/27 17 | 0.0367 | 0.1032 | 0.0172 | 0.0642 | 0.2179 | 0.0230 | 0.1795 | 0.3386 | 0.0548 | 0.1130 | 0.0160 | 1.164 |
| NH61/27 38 | 0.0381 | 0.0955 | 0.0142 | 0.0602 | 0.2085 | 0.0188 | 0.1744 | 0.3129 | 0.0412 | 0.0989 | 0.0163 | 1.079 |
| NH61/27 31 | 0.0366 | 0.0887 | 0.0151 | 0.0492 | 0.1914 | 0.0209 | 0.1817 | 0.3285 | 0.0407 | 0.1017 | 0.0172 | 1.072 |
| NH61/27 9 | 0.0360 | 0.1020 | 0.0113 | 0.0462 | 0.1843 | 0.0183 | 0.1769 | 0.3123 | 0.0406 | 0.0867 | 0.0228 | 1.037 |
| NH61/27 1 | 0.0228 | 0.0676 | 0.0105 | 0.0397 | 0.1595 | 0.0146 | 0.1672 | 0.3287 | 0.0477 | 0.0932 | 0.0169 | 0.968 |
| NH61/27 15 | 0.0292 | 0.0719 | 0.0082 | 0.0378 | 0.1555 | 0.0137 | 0.1836 | 0.3148 | 0.0356 | 0.0912 | 0.0185 | 0.960 |
| NH61/27 24 | 0.0253 | 0.0682 | 0.0088 | 0.0360 | 0.1378 | 0.0137 | 0.1642 | 0.3240 | 0.0411 | 0.0956 | 0.0178 | 0.932 |
| NH61/27 29 | 0.0240 | 0.0679 | 0.0099 | 0.0394 | 0.1444 | 0.0147 | 0.1654 | 0.3111 | 0.0390 | 0.0950 | 0.0161 | 0.927 |
| NH61/27 27 | 0.0282 | 0.0715 | 0.0116 | 0.0388 | 0.1541 | 0.0168 | 0.1635 | 0.2855 | 0.0399 | 0.0962 | 0.0147 | 0.921 |
| NH61/27 10 | 0.0268 | 0.0692 | 0.0106 | 0.0383 | 0.1633 | 0.0174 | 0.1658 | 0.2897 | 0.0373 | 0.0826 | 0.0172 | 0.918 |
| NH61/27 37 | 0.0289 | 0.0632 | 0.0100 | 0.0371 | 0.1485 | 0.0148 | 0.1595 | 0.2978 | 0.0415 | 0.0948 | 0.0170 | 0.913 |
| NH61/27 19 | 0.0203 | 0.0540 | 0.0093 | 0.0327 | 0.1388 | 0.0127 | 0.1399 | 0.3062 | 0.0453 | 0.0878 | 0.0145 | 0.861 |
| NH61/27 21 | 0.0090 | 0.0286 | 0.0042 | 0.0157 | 0.0613 | 0.0077 | 0.1070 | 0.2187 | 0.0381 | 0.0811 | 0.0156 | 0.587 |
| NH61/27 32 | 0.0094 | 0.0217 | 0.0037 | 0.0131 | 0.0580 | 0.0080 | 0.1037 | 0.2249 | 0.0377 | 0.0844 | 0.0125 | 0.577 |
| NH61/27 33 | 0.0091 | 0.0279 | 0.0031 | 0.0135 | 0.0522 | 0.0062 | 0.1035 | 0.2099 | 0.0361 | 0.0773 | 0.0153 | 0.554 |
| NH61/27 14 | 0.0094 | 0.0268 | 0.0037 | 0.0137 | 0.0582 | 0.0066 | 0.0914 | 0.1975 | 0.0343 | 0.0680 | 0.0136 | 0.523 |
| NH61/27 7 | 0.0070 | 0.0339 | 0.0028 | 0.0099 | 0.0496 | 0.0062 | 0.0820 | 0.1939 | 0.0343 | 0.0589 | 0.0188 | 0.497 |
| SR1 4 | 0.0084 | 0.0440 | 0.0023 | 0.0093 | 0.0461 | 0.0060 | 0.0767 | 0.1565 | 0.0339 | 0.0497 | 0.0261 | 0.459 |
| SR1 5 | 0.0067 | 0.0427 | 0.0020 | 0.0092 | 0.0408 | 0.0061 | 0.0724 | 0.1452 | 0.0368 | 0.0521 | 0.0239 | 0.438 |
| SR1 7 | 0.0082 | 0.0370 | 0.0019 | 0.0082 | 0.0380 | 0.0054 | 0.0706 | 0.1401 | 0.0324 | 0.0462 | 0.0230 | 0.411 |
| SR1 10 | 0.0052 | 0.0392 | 0.0020 | 0.0079 | 0.0318 | 0.0056 | 0.0642 | 0.1431 | 0.0373 | 0.0504 | 0.0226 | 0.409 |
| SR1 2 | 0.0066 | 0.0358 | 0.0015 | 0.0070 | 0.0362 | 0.0051 | 0.0663 | 0.1333 | 0.0312 | 0.0430 | 0.0226 | 0.389 |
| Average | 0.0070 | 0.0398 | 0.0020 | 0.0083 | 0.0386 | 0.0056 | 0.0700 | 0.1436 | 0.0343 | 0.0483 | 0.0237 | 0.421 |
| NH19/27 3 | 0.0084 | 0.0238 | 0.0027 | 0.0086 | 0.0487 | 0.0059 | 0.0868 | 0.1941 | 0.0430 | 0.0688 | 0.0163 | 0.507 |
| NH19/27 1 | 0.0076 | 0.0257 | 0.0030 | 0.0111 | 0.0469 | 0.0058 | 0.0855 | 0.1894 | 0.0428 | 0.0671 | 0.0151 | 0.500 |
| NH19/27 7 | 0.0087 | 0.0192 | 0.0021 | 0.0085 | 0.0436 | 0.0051 | 0.0821 | 0.1922 | 0.0427 | 0.0717 | 0.0135 | 0.489 |
| NH19/27 6 | 0.0041 | 0.0125 | 0.0012 | 0.0054 | 0.0212 | 0.0032 | 0.0498 | 0.1646 | 0.0503 | 0.0617 | 0.0104 | 0.385 |

TABLE 10
Sterol Analysis of mature seed from Brassica napus transformed with N-truncated
Hevea HMGR and N. tabacum SMT1 (MH7)
Total sterols as % of dry weight

| | | | | | 24 | d7- | | | | | brassica | | |
|---|---|---|---|---|---|---|---|---|---|---|---|---|---|
| Smpl code | squalene | cycloart | 24 mca | 24 mloph | eloph | avena | isofuc | sito | stig | camp | sterol | chol | Total |
| MH7 11a | 0.0024 | 0.0073 | 0.0035 | 0.0244 | 0.0033 | 0.0015 | 0.0028 | 0.2219 | 0.0022 | 0.0791 | 0.0233 | 0.0020 | 0.374 |
| MH7 170 | 0.0024 | 0.0050 | 0.0000 | 0.0042 | 0.0000 | 0.0000 | 0.0024 | 0.2185 | 0.0023 | 0.0951 | 0.0270 | 0.0029 | 0.360 |
| MH7 15a | 0.0019 | 0.0039 | 0.0000 | 0.0054 | 0.0000 | 0.0000 | 0.0013 | 0.1683 | 0.0016 | 0.0717 | 0.0289 | 0.0014 | 0.284 |
| MH7 14a | 0.0022 | 0.0041 | 0.0000 | 0.0087 | 0.0000 | 0.0000 | 0.0018 | 0.1714 | 0.0028 | 0.0590 | 0.0238 | 0.0031 | 0.277 |
| Control | 0.0031 | 0.0052 | 0.0034 | 0.0177 | 0.0027 | 0.0000 | 0.0021 | 0.1327 | 0.0036 | 0.0475 | 0.0230 | 0.0017 | 0.243 |

1. The use of a gene expressing a non-feed back inhibited HMG-reductase in combination with a gene expressing sterol methyltransferasel to increase the level of sterols in plants.

2. The use according to claim 1, wherein the level of 4-desmethylsterols is increased in the plants by at least 10%.

3. The use according to claim 1, wherein the sterols are increased in seeds, more preferred in oilseeds.

4. The use according to claim 3, wherein the seeds are from tobacco, canola, sunflower, rape, soy or peanut.

5. The use according to claim 1, wherein the non feedback inhibited HMG-reductase is expressed by a truncated non-plant HMG gene.

6. The use according to claim 5, wherein the HMG-reductase expressed by the truncated HMG-reductase gene lacks the membrane-binding domain.

7. The use according to claim 1, wherein the non-feedback inhibited HMG-reductase is expressed by a truncated plant HMG-reductase gene.

8. The use according to claim 1, wherein the HMG-reductase can be derived from Asteraceae.

9. The use according to claim 8, wherein the HMGR gene can be derived from Hevea brasiliensis or the HMGR gene is a truncated version of a gene which can be derived from Hevea brasiliensis.

10. Use according to claim 9, wherein the HMGR gene is the hmg 1 gene derived from Hevea brasiliensis or a truncated version of said gene.

11. A method of transforming a plant by
A1) transforming a plant cell with a recombinant DNA construct comprising a DNA segment encoding a polypeptide with non feedback inhibited HMGR activity and a polypeptide encoding a sterol methyltransferasel activity and promoters for driving the expression of said polypeptides in said plant cell to form a transformed plant cell; or
A2) re-transforming a plant cell expressing a non-feedback inhibited HMGR activity with a gene encoding a sterol methyltransferasel activity; or
A3) re-transforming a plant cell expressing a sterol methyltransferasel activity with a gene encoding a non-feedback inhibited HMGR activity; and
D) regenerating the above transformed plant cells into transgenic plants; and
E) selecting transgenic plants that have enhanced levels of 4-desmethylsterols compared to wild type strains of the same plant.

12. Plant obtainable by a method according to claim 11.

13. Plant tissue obtained from a plant according to claim 12.

14. Plant tissue according to claim 13, selected from the group of leaves, fruit and seeds.

15. Plant having incorporated in its genome a heterologous gene encoding a non-feed back inhibited HMGR activity in combination with an heterologous gene encoding SMT1.

16. Plant according to claim 15 wherein the gene encoding a non-feed back inhibited HMGR activity is a gene encoding a truncated polypeptide HMGR activity.
